(19) United States
(12) Patent Application Publication
(10) Pub. No.: US 2021/0177284 A1
(43) Pub. Date: Jun. 17, 2021

(54) MEDICAL OBSERVATION SYSTEM, MEDICAL OBSERVATION APPARATUS, AND METHOD FOR DRIVING MEDICAL OBSERVATION APPARATUS

(71) Applicant: Sony Corporation, Tokyo (JP)

(72) Inventors: Tetsuro KUWAYAMA, Tokyo (JP); Takanori FUKAZAWA, Tokyo (JP); Takeshi MATSUI, Tokyo (JP)

(73) Assignee: Sony Corporation, Tokyo (JP)

(21) Appl. No.: 17/252,744

(22) PCT Filed: Jun. 21, 2019
(86) PCT No.: PCT/JP2019/024775

§ 371 Date: Dec. 16, 2020

(30) Foreign Application Priority Data
Jul. 2, 2018 (JP) 2018-125829

(51) Int. Cl. A61B 5/026; G16H 20/40; G16H 30/40; A61B 1/06; G02B 21/00; G02B 21/18

(52) U.S. Cl. CPC A61B 5/0261; G16H 20/40; G16H 30/40; G02B 21/18; A61B 1/0684; G02B 21/0012; A61B 1/063

(57) ABSTRACT

A medical observation system (2, 3) includes: a light source (223) configured to illuminate an affected body part; a branching optical system (213) configured to separate light from the affected body part into a plurality of polarized lights having different polarization directions; a detection unit (313) configured to individually detect the plurality of polarized lights; an arithmetic operation unit (305) configured to individually calculate speckle contrasts on the basis of detection results of the plurality of polarized lights; and a processing unit (303) configured to execute processing with respect to observation of the affected body part on the basis of at least any of calculation results of the speckle contrasts corresponding to the plurality of polarized lights.

### TECHNICAL FIELD

The present disclosure relates to a medical observation system, a medical observation apparatus, and a method for driving the medical observation apparatus.

### BACKGROUND ART

With the recent development of technology pertaining to observation of affected body parts, represented by an operating microscope, endoscope, and the like, more targets may be observed. In particular, various technologies for allowing observation of a blood flow also have been proposed in recent years.

As technologies for observing body parts with a motion such as a blood flow, a technology using speckles generated according to radiation of light to an affected body part that is an observation target is conceivable, and particularly, a technology using a speckle contrast is attracting attention. A speckle contrast is a value calculated in response to a light intensity distribution and has characteristics in which the value increases in a body part without a motion and decreases in a body part with a motion. By using such characteristics, identification of a body part with a motion, recognition of the magnitude of the amount of the motion, and the like may be performed by evaluating a speckle contrast. For example, Patent Literature 1 discloses an example of a technology for allowing observation of a body part with a motion such as a blood flow with high accuracy using a speckle contrast.

### CITATION LIST

### Patent Literature

### [PTL 1]

JP 2016-151524A

### SUMMARY

### Technical Problem

Meanwhile, when an affected body part that is an observation target has a slight motion, there are cases in which it is difficult to detect the motion. For example, if an affected body part has a slight motion, even when a speckle contrast is used to observe the affected body part, there are cases in which change in the speckle contrast tends to decrease and thus it is difficult to detect the motion. Further, in a situation where the womb of a patient is observed, the amount of light that may be condensed by an imaging unit or the like to acquire an image of an affected body part is limited and thus a system capable of using condensed light with high efficiency is needed.

Accordingly, the present disclosure proposes a technology for realizing observation of an affected body part with motion in a more suitable state.

### Solution to Problem

According to the present disclosure, there is provided a medical observation system including: a light source configured to illuminate an affected body part; a branching optical system configured to separate light from the affected body part into a plurality of polarized lights having different polarization directions; a detection unit configured to individually detect the plurality of polarized lights; an arithmetic operation unit configured to individually calculate speckle contrasts on the basis of detection results of the plurality of polarized lights; and a processing unit configured to execute processing with respect to observation of the affected body part on the basis of at least any of calculation results of the speckle contrasts corresponding to the plurality of polarized lights.

Furthermore, according to the present disclosure, there is provided a medical observation apparatus including: a branching optical system configured to separate light from an affected body part into a plurality of polarized lights having different polarization directions; a detection unit configured to individually detect the plurality of polarized lights; an arithmetic operation unit configured to individually calculate speckle contrasts on the basis of detection results of the plurality of polarized lights; and a processing unit configured to execute processing with respect to observation of the affected body part on the basis of at least any of calculation results of the speckle contrasts corresponding to the plurality of polarized lights.

Furthermore, according to the present disclosure, there is provided a medical observation apparatus including: an arithmetic operation unit configured to individually calculate speckle contrasts on the basis of detection results of a plurality of polarized lights having different polarization directions, separated from light from an affected body part; and a processing unit configured to execute processing with respect to observation of the affected body part on the basis of at least any of calculation results of the speckle contrasts corresponding to the plurality of polarized lights.

Furthermore, according to the present disclosure, there is provided a method for driving a medical observation apparatus, using a computer, including:

individually calculating speckle contrasts on the basis of detection results of a plurality of polarized lights having different polarization directions, separated from light from an affected body part; and executing processing with respect to observation of the affected body part on the basis of at least any of calculation results of the speckle contrasts corresponding to the plurality of polarized lights.

### Advantageous Effects of Invention

According to the present disclosure as described above, a technology for realizing observation of an affected body part with a motion in a more suitable state is provided.

The aforementioned effects are not necessarily limitative and any effect described in this specification or other effects that may be ascertained from this specification may be obtained in addition to or instead of the aforementioned effects.

### BRIEF DESCRIPTION OF DRAWINGS

FIG. 1 is a diagram illustrating an example of a schematic configuration of an endoscopic operation system to which the technology according to the present disclosure is applied.

FIG. 2 is a block diagram illustrating an example of a functional configuration of a camera head and a CCU illustrated in FIG. 1.

FIG. 3 is an explanatory diagram for describing an overview of a speckle contrast.

FIG. 4 is an explanatory diagram for describing an overview of a speckle contrast.

FIG. 5 is an explanatory diagram for describing an example of a relationship between a speckle contrast and a motion of an object.

FIG. 6 is an explanatory diagram for describing an influence on speckle contrast calculation results when polarized light is used.

FIG. 7 is a diagram illustrating examples of images having different speckle contrast levels.

FIG. 8 is an explanatory diagram for describing another example of a relationship between a speckle contrast and a motion of an object.

FIG. 9 is an explanatory diagram for describing the basic concept of a technology pertaining to observation of affected body parts in a medical observation system according to an embodiment of the present disclosure.

FIG. 10 is an explanatory diagram for describing an example of a configuration of the medical observation system according to the embodiment.

FIG. 11 is a block diagram illustrating an example of a functional configuration of the medical observation system according to the embodiment.

FIG. 12 is a flowchart illustrating an example of a flow of a series of processes of the medical observation system according to the embodiment.

FIG. 13 is an explanatory diagram for describing an overview of a medical observation system according to modified example 1.

FIG. 14 is an explanatory diagram for describing an overview of a medical observation system according to modified example 2.

FIG. 15 is an explanatory diagram for describing an example of processing of a medical observation system according to modified example 4.

FIG. 16 is a functional block diagram illustrating a configuration example of a hardware configuration of an information processing apparatus constituting the medical observation system according to an embodiment of the present disclosure.

FIG. 17 is an explanatory diagram for describing an application example of the medical observation system according to an embodiment of the present disclosure.

### DESCRIPTION OF EMBODIMENTS

Hereinafter, suitable embodiments of the present disclosure will be described in detail with reference to the attached drawings. Meanwhile, components having substantially the same functional configuration are denoted by the same sign and redundant description thereof is omitted in this specification and drawings.

Further, it is assumed that description is performed in the following order.

1. Configuration example of medical observation system
2. Examination with respect to observation using speckle
3. Technical features
3.1. Basic concept
3.2. Configuration example of system
3.3. Functional configuration

### 3.4. Processing

3.5. Modified examples
3.6. Operation effects

### 3.7. Supplement

4. Example of hardware configuration
5. Application example

### 6. Conclusion

### 1. CONFIGURATION EXAMPLE OF MEDICAL OBSERVATION SYSTEM

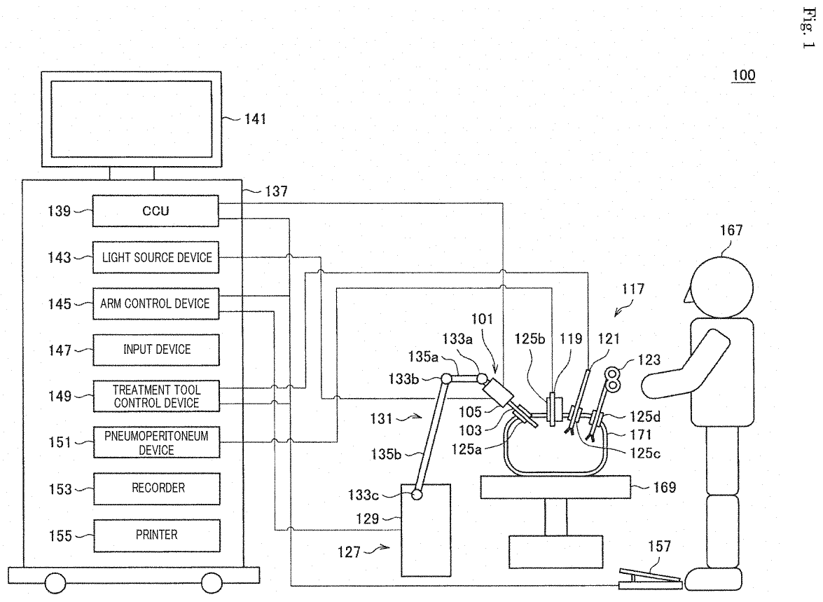

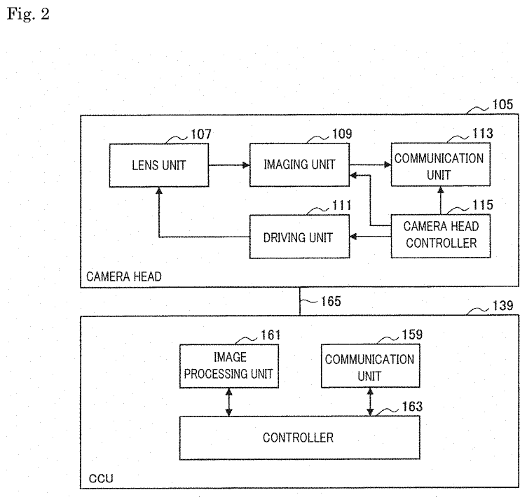

First, an example of a so-called endoscopic operation system will be described as an example of a schematic configuration of a medical observation system to which a technology of an embodiment of the present disclosure is applicable with reference to FIG. 1 and FIG. 2.

For example, FIG. 1 is a diagram illustrating an example of the schematic configuration of the endoscopic observation system to which a technology of the present disclosure is applicable. FIG. 1 illustrates a state in which an operator (doctor) 167 performs an operation on a patient 171 on a patient bed 169 using an endoscopic operation system 100. As illustrated, the endoscopic operation system 100 includes an endoscope 101, other operation instruments 117, a supporting arm device 127 for supporting the endoscope 101, and a cart 137 on which various devices for an endoscopic operation are mounted.

In an endoscopic operation, a plurality of tubular perforating tools called trocars 125a to 125d puncture the abdominal wall without cutting the abdominal wall open. Then, a barrel 103 of the endoscope 101 and other operation instruments 117 are inserted into the body cavity of the patient 171 from the trocars 125a to 125d. In the illustrated example, a pneumoperitoneum tube 119, an energy treatment tool 121, and forceps 123 are inserted into the body cavity of the patient 171 as the other operation instruments 117. In addition, the energy treatment tool 121 is a treatment tool that performs dissection and separation of tissues or blood vessel sealing and the like using high-frequency current or ultrasonic vibration. However, the illustrated operation instruments 117 are merely an example and various operation instruments used in general endoscopic operations, such as a pincette and a retractor may be used as the operation instruments 117.

An image of an operation site in the body cavity of the patient 171, captured by the endoscope 101, is displayed on a display device 141. The operator 167 performs treatment such as dissection of an affected body part, for example, using the energy treatment tool 121 and the forceps 123 while viewing the image of the operation site displayed on the display device 141 in real time. Although not illustrated, the pneumoperitoneum tube 119, the energy treatment tool 121, and the forceps 123 are supported by the operator 167, an assistant, or the like during operation.

(Supporting Arm Device)

The supporting arm device 127 includes an arm part 131 extending from a base part 129. In the illustrated example, the arm part 131 includes joints 133a, 133b and 133c, and links 135a and 135b and is driven according to control of an arm control device 145. The endoscope 101 is supported by the arm part 131 and the position and posture thereof are controlled. Accordingly, stable position fixing of the endoscope 101 may be realized.

(Endoscope)

The endoscope 101 includes the barrel 103 with an area having a predetermined length from the front end thereof, which is inserted into the body cavity of the patient 171, and a camera head 105 connected to the base end of the barrel 103. Although the endoscope 101 configured as a so-called hard mirror having the hard barrel 103 is illustrated in the illustrated example, the endoscope 101 may be configured as a so-called flexible mirror having a flexible barrel 103. Meanwhile, the camera head 105 or the endoscope 101 including the camera head 105 corresponds to an example of a “medical observation apparatus.”

An opening into which an objective lens is fitted is provided at the front end of the barrel 103. A light source device 143 is connected to the endoscope 101, and light generated from the light source device 143 is guided to the front end of the barrel through a light guide extending in the barrel 103 and radiated to an observation target (in other words, an imaging target) in the body cavity of the patient 171 through the objective lens. Meanwhile, the endoscope 101 may be a straight view mirror, an oblique view mirror, or a side view mirror.

An optical system and an imaging element are provided in the camera head 105, and light from the observation target (observation light) is condensed on the imaging element by the optical system. The observation light is photoelectrically converted by the imaging element to generate an electrical signal corresponding to the observation light, that is, an image signal corresponding to an observation image. The image signal is transmitted to a camera control unit (CCU) 139 as raw data. Meanwhile, the camera head 105 is provided with a function of adjusting a magnification and a focal distance by appropriately driving the optical system.

Meanwhile, the camera head 105 may be equipped with a plurality of imaging elements in order to cope with stereoscopic vision (3D display) and the like, for example. In this case, a plurality of relay optical systems may be provided in the barrel 103 in order to guide observation light to each of the plurality of imaging elements.

(Various Devices Mounted on Cart)

The CCU 139 is configured as a central processing unit (CPU), a graphics processing unit (GPU), or the like and integrally controls operations of the endoscope 101 and the display device 141. Specifically, the CCU 139 performs, on an image signal received from the camera head 105, various types of image processing for displaying an image based on the image signal, such as developing processing (demosaic processing), for example. The CCU 139 provides the image signal on which the image processing has been performed to the display device 141. Further, the CCU 139 transmits a control signal to the camera head 105 and controls operation thereof. The control signal may include information about imaging conditions such as a magnification and a focal distance.

The display device 141 displays an image based on the image signal on which image processing has been performed by the CCU 139 according to control of the CCU 139. In a case in which the endoscope 101 handles imaging with high resolution such as 4K (number of horizontal pixels, 3840×number of vertical pixels, 2160) or 8K (number of horizontal pixels, 7680×number of vertical pixels, 4320), for example, and/or a case in which it corresponds to 3D display, a display device capable of performing high-resolution display and/or a display device capable of performing 3D display may be used as the display device 141 for the respective cases. When the endoscope 101 is able to handle imaging with high resolution such as 4K or 8K, a more immersive feeling is obtained by using a display device having a size of 55 inches or more as the display device 141. In addition, a plurality of display devices 141 having different resolutions and sizes may be provided according to applications.

The light source device 143 is composed of, for example, light sources such as light emitting diodes (LEDs) and supplies light to be radiated when an operation site is imaged to the endoscope 101.

The arm control device 145 is composed of, for example, a processor such as a CPU and operates according to a predetermined program to control operation of the arm part 131 of the supporting arm device 127 according to a predetermined control method.

An input device 147 is an input interface for the endoscopic operation system 100. A user may input various types of information and instructions to the endoscopic operation system 100 through the input device 147. For example, the user may input various types of information about an operation, such as body information of a patient and an operation method through the input device 147. In addition, the user may input, for example, an instruction for driving the arm part 131, an instruction for changing imaging conditions (a type of radiated light, a magnification, a focal distance, and the like) of the endoscope 101, an instruction for driving the energy treatment tool 121, and the like through the input device 147.

The type of the input device 147 is not limited and the input device 147 may be various known input devices. As the input device 147, for example, a mouse, a keyboard, a touch panel, a switch, a foot switch 157, a lever and/or the like may be applied. When a touch panel is used as the input device 147, the touch panel may be provided on the display surface of the display device 141.

Alternatively, the input device 147 may be, for example, a device worn by a user, such as a glasses type wearable device or a head mounted display (HMD), and various inputs may be performed in response to gestures and sight lines of the user detected by these devices. In addition, the input device 147 may include a camera capable of detecting a motion of a user, and various inputs may be performed in response to gestures and sight lines of the user detected from a video captured by the camera. Further, the input device 147 may include a microphone capable of receiving speech of a user, and various inputs may be performed in response to speech via the microphone. In this manner, the input device 147 is configured such that various types of information may be input thereto in a contactless manner so that a user (e.g., the operator 167) belonging to a clean area, particularly, may operate an apparatus belonging to an unclean area in a contactless manner. Further, the user may operate the apparatus without taking his/her hands off from operation equipment held by him/her and thus user convenience is improved.

A treatment tool control device 149 controls operation of the energy treatment tool 121 for cauterization and dissection of tissues or blood vessel sealing and the like. A pneumoperitoneum device 151 feeds a gas into the body cavity of the patient 171 through the pneumoperitoneum tube 119 in order to inflate the body cavity for the purpose of securing a view for the endoscope 101 and securing a work space of an operator. A recorder 153 is a device capable of recording various types of information about operations. A printer 155 is a device capable of printing various types of information about operations in various forms such as text, images and graphs.

Hereinafter, a particularly characteristic configuration of the endoscopic operation system 100 will be described in more detail.

(Supporting Arm Device)

The supporting arm device 127 includes the base part 129 that is a base, and the arm part 131 extending from the base part 129. Although the arm part 131 includes the plurality of joints 133a, 133b and 133c and the plurality of links 135a and 135b connected by the joint 133b in the illustrated example, FIG. 1 illustrates the configuration of the arm part 131 in a simplified manner for simplification. In practice, shapes, numbers and arrangement of the joints 133a to 133c and the links 135a and 135b and directions of rotation axes of the joints 133a to 133c, and the like may be appropriately set such that the arm part 131 has a desired degree of freedom. For example, the arm part 131 may be suitably configured to have a degree of freedom equal to or greater than 6 degrees of freedom. Accordingly, the endoscope 101 may be freely moved in a range of a motion of the arm part 131 and thus the barrel 103 of the endoscope 101 may be inserted into the body cavity of the patient 171 in a desired direction.

The joints 133a to 133c are provided with actuators and configured such that they can rotate on a predetermined rotation axis according to operations of the actuators. The operations of the actuators are controlled by the arm control device 145 so that rotation angles of the joints 133a to 133c are controlled and the operation of the arm part 131 is controlled. Accordingly, control of the position and the posture of the endoscope 101 may be realized. Here, the arm control device 145 may control the operation of the arm part 131 through various known control methods such as force control and position control.

For example, the operator 167 may perform an appropriate operation input through the input device 147 (including the foot switch 157) such that the operation of the arm part 131 is appropriately controlled by the arm control device 145 in response to the operation input to control the position and the posture of the endoscope 101. According to this control, the endoscope 101 at the front end of the arm part 131 may be moved from an arbitrary position to an arbitrary position and then fixed and supported at the position after the movement. Meanwhile, the arm part 131 may be operated through a master-slave method. In this case, the arm part 131 may be remotely operated by a user through the input device 147 provided in a place separated from an operating room.

In addition, in a case where force control is applied, the arm control device 145 may perform so-called power assist control of receiving an external force from a user and driving the actuators of the joints 133a to 133c such that the arm part 131 is smoothly moved in response to the external force. Accordingly, a user is able to move the arm part 131 with a relatively weak force when moving the arm part 131 in direct contact with the arm part 131. Therefore, it is possible to move the endoscope 101 more intuitively through an easier operation to improve user convenience.

Here, the endoscope 101 is generally supported by a doctor called a scopist in an endoscopic operation. In contrast, the position of the endoscope 101 may be fixed more securely without manpower by using the supporting arm device 127, and thus an image of an operation site may be stably obtained and operation may be smoothly performed.

Meanwhile, the arm control device 145 need not necessarily provided in the cart 137. Further, the arm control device 145 need not necessarily be a single device. For example, the arm control device 145 may be provided at each of the joints 133a to 133c of the arm part 131 of the supporting arm device 127 or operation control of the arm part 131 may be realized by a plurality of arm control devices 145 in cooperation.

(Light Source Device)

The light source device 143 supplies light to be radiated to image an operation site to the endoscope 101. The light source device 143 is configured as, for example, a white light source composed of LEDs, laser light sources or a combination thereof. Here, in a case where the white light source is configured as a combination of RGB laser light sources, white balance of a captured image may be adjusted in the light source device 143 because in this case an output intensity and an output timing of each color (each wavelength) may be controlled with high accuracy. In addition, images corresponding to RGB may be captured in a time division manner by radiating laser light from the RGB laser light sources to an observation target in a time division manner and controlling the operation of the imaging element of the camera head 105 in synchronization with the radiation timing. According to this method, a color image may be acquired without providing a color filter in the imaging element.

In addition, the operation of the light source device 143 may be controlled such that the intensity of output light thereof changes at each of predetermined times. It is possible to generate a high-dynamic range image without black defects and flared highlights by controlling the operation of the imaging element of the camera head 105 in synchronization with timings at which the intensity of light changes to acquire images in a time division manner and combining the images.

Further, the light source device 143 may be configured to be able to supply light in a predetermined wavelength band corresponding to special light observation. In the special light observation, imaging of predetermined tissues such as blood vessels of the mucous membrane surface layer with a high contrast, so-called narrow band imaging, is performed by radiating light in a narrower band than radiation light (i.e., white light) in normal observation, using a wavelength dependence of absorption of light in a body tissue. Alternatively, in the special light observation, fluorescence observation for obtaining an image using fluorescence generated by radiating excited light may be performed. In the fluorescence observation, an operation of radiating excited light to a body tissue and observing fluorescence from the body tissue (self-fluorescence observation) or an operation of locally injecting a reagent such as indocyanine green (ICG) to the body tissue and radiating excited light corresponding to the fluorescence wavelength of the reagent to the body tissue to obtain a fluorescent image may be performed. The light source device 143 may be configured to be able to supply narrow-band light and/or excited light corresponding to the special light observation.

(Camera Head and CCU)

The functions of the camera head 105 of the endoscope 101 and the CCU 139 will be described in more detail with reference to FIG. 2. FIG. 2 is a block diagram illustrating an example of functional configurations of the camera head 105 and the CCU 139 illustrated in FIG. 1.

Referring to FIG. 2, the camera head 105 includes a lens unit 107, an imaging unit 109, a driving unit 111, a communication unit 113, and a camera head controller 115 as functions thereof. In addition, the CCU 139 includes a communication unit 159, an image processing unit 161, and a controller 163 as functions thereof. The camera head 105 and the CCU 139 are connected through a transmission cable 165 such that they can bidirectionally communicate with each other.

First, the functional configuration of the camera head 105 will be described. The lens unit 107 is an optical system provided at a connecting part with the barrel 103. Observation light from the front end of the barrel 103 is guided to the camera head 105 and incident to the lens unit 107. The lens unit 107 is configured as a combination of a plurality of lenses including a zoom lens and a focus lens. The optical properties of the lens unit 107 are adjusted such that observation light is condensed on a light-receiving surface of an imaging element of the imaging unit 109. In addition, the zoom lens and the focus lens are configured such that the positions thereof on an optical axis are movable for adjustment of a magnification and a focus of a captured image.

The imaging unit 109 is composed of the imaging element and disposed behind the lens unit 107. Observation light that has passed through the lens unit 107 is condensed on the light-receiving surface of the imaging element and an image signal corresponding to the observation image is generated through photoelectric conversion. The image signal generated by the imaging unit 109 is provided to the communication unit 113.

As the imaging element constituting the imaging unit 109, for example, a complementary metal oxide semiconductor (CMOS) type image sensor having a Bayer arrangement and capable of color imaging is used. Meanwhile, an imaging element capable of capturing images with a high resolution of 4K or higher may be used as the imaging element, for example. By obtaining an image of the operation site in high resolution, the operator 167 can ascertain the state of the operation site in more detail and thus can perform the operation more smoothly.

In addition, the imaging element constituting the imaging unit 109 may be configured to have a pair of imaging elements for acquiring image signals for the right eye and the left eye corresponding to 3D display. When 3D display is performed, the operator 167 can ascertain the depth of the body tissue in the operation site more accurately. Meanwhile, when the imaging unit 109 is configured as a multi-plate type, a plurality of types of lens unit 107 are also provided to correspond to each imaging element.

Further, the imaging unit 109 need not necessarily be provided in the camera head 105. For example, the imaging unit 109 may be provided immediately behind the objective lens inside the barrel 103.

The driving unit 111 is composed of actuators and moves the zoom lens and the focus lens of the lens unit 107 along the optical axis by a predetermined distance according to control of the camera head controller 115. Accordingly, a magnification and a focus of an image captured by the imaging unit 109 may be appropriately adjusted.

The communication unit 113 is configured as a communication device for transmitting/receiving various types of information to/from the CCU 139. The communication unit 113 transmits an image signal obtained from the imaging unit 109 to the CCU 139 as raw data through the transmission cable 165. Here, it is desirable that the image signal be transmitted through optical communication in order to display a captured image of the operation site with low latency. This is because a moving image of the operation site needs to be displayed in real time as long as possible for more stable and reliable operation because the operator 167 performs the operation while observing a state of an affected body part through a captured image. In a case where optical communication is performed, the communication unit 113 is provided with a photoelectric conversion module for converting an electrical signal into an optical signal. The image signal is converted into an optical signal through the photoelectric conversion module and then transmitted to the CCU 139 through the transmission cable 165.

In addition, the communication unit 113 receives a control signal for controlling the operation of the camera head 105 from the CCU 139. The control signal may include, for example, information about imaging conditions such as information for designating a frame rate of a captured image, information for designating an exposure value during imaging, and/or information for designating a magnification and a focus of the captured image. The communication unit 113 provides the received control signal to the camera head controller 115. Meanwhile, the control signal from the CCU 139 may be transmitted through optical communication. In this case, the communication unit 113 is provided with a photoelectric conversion module for converting an optical signal into an electrical signal, and the control signal is converted into an electrical signal through the photoelectric conversion module and then provided to the camera head controller 115.

Meanwhile, the aforementioned imaging conditions such as a frame rate, an exposure value, a magnification, and a focus are automatically set by the controller 163 of the CCU 139 on the basis of the acquired image signal. That is, the endoscope 101 is equipped with a so-called auto exposure (AE) function, an auto focus (AF) function and an auto white balance (AWB) function.

The camera head controller 115 controls the operation of the camera head 105 on the basis of a control signal from the CCU 139 received through the communication unit 113. For example, the camera head controller 115 controls the operation of the imaging element of the imaging unit 109 on the basis of information for designating a frame rate of a captured image and/or information for designating exposure during imaging. In addition, the camera head controller 115, for example, appropriately moves the zoom lens and the focus lens of the lens unit 107 through the driving unit 111 on the basis of information for designating a magnification and a focus of a captured image. Further, the camera head controller 115 may have a function of storing information for identifying the barrel 103 and the camera head 105.

Meanwhile, components such as the lens unit 107 and the imaging unit 109 may be disposed in a closed structure having high airtightness and waterproofness such that the camera head 105 has a resistance to autoclave sterilization processing.

Next, the functional configuration of the CCU 139 will be described. The communication unit 159 is configured as a communication device for transmitting/receiving various types of information to/from the camera head 105. The communication unit 159 receives, from the camera head 105, an image signal transmitted through the transmission cable 165. Here, the image signal may be transmitted through suitable optical communication, as described above. In this case, a photoelectric conversion module for converting an optical signal into an electrical signal is provided in the communication unit 159 for optical communication. The communication unit 159 provides the image signal converted into an electrical signal to the image processing unit 161.

In addition, the communication unit 159 transmits a control signal for controlling the operation of the camera head 105 to the camera head 105. The control signal may also be transmitted through optical communication.

The image processing unit 161 performs various types of image processing on the image signal that is raw data transmitted from the camera head 105. The image processing may include, for example, various types of known signal processing such as developing processing, quality-enhancement processing (band emphasis processing, superresolution processing, noise reduction (NR) processing and/or image stabilization processing, etc.) and/or enlargement processing (electronic zoom processing). In addition, the image processing unit 161 performs detection processing on the image signal for executing AE, AF and AWB.

The image processing unit 161 is configured as a processor such as a CPU or a GPU, and the aforementioned image processing and detection processing may be performed through the operation of the processor according to a predetermined program. Meanwhile, in a case where the image processing unit 161 is configured as a plurality of GPUs, the image processing unit 161 appropriately divides information about an image signal and performs image processing in parallel through the plurality of GPUs.

The controller 163 performs various types of control with respect to imaging of the operation site through the endoscope 101 and display of an image captured by the imaging. For example, the controller 163 may generate a control signal for controlling the operation of the camera head 105. Here, in a case in which imaging conditions are input by a user, the controller 163 generates the control signal on the basis of the user input. Alternatively, in a case where the endoscope 101 has the AE function, the AF function and the AWB function, the controller 163 appropriately calculates an optimal exposure value, focal distance and white balance in response to a result of detection processing performed by the image processing unit 161 and generates the control signal.

Further, the controller 163 causes the display device 141 to display an image of the operation site on the basis of the image signal on which image processing has been performed by the image processing unit 161. Here, the controller 163 recognizes various objects in the image of the operation site using various image recognition technologies. For example, the controller 163 may recognize operation tools such as forceps, a specific bio-part, bleeding, mist when the energy treatment tool 121 is used, and the like by detecting shapes, colors and the like of edges of objects included in the image of the operation site. The controller 163 causes various types of operation assistance information to be superposed on the image of the operation site using the recognition result when the display device 141 displays the image of the operation site. By displaying the operation assistance information in a superposed manner and presenting it to the operator 167, the operation may be performed more safely and securely.

The transmission cable 165 for connecting the camera head 105 and the CCU 139 is an electrical signal cable for electrical signal communication, an optical fiber for optical communication or a composite cable thereof.

Here, although communication is performed in a wired manner using the transmission cable 165 in the illustrated example, communication between the camera head 105 and the CCU 139 may be performed in a wireless manner. In a case where communication therebetween is performed in a wireless manner, a situation in which movement of medical staffs in the operating room is obstructed by the transmission cable 165 may be resolved because the transmission cable 165 need not be installed in the operating room.

An example of the endoscopic operation system 100 to which the technology of the present disclosure is applicable has been described above. Although the endoscopic operation system 100 has been described as an example here, the system to which the technology of the present disclosure is applicable is not limited to such an example. For example, the technology of the present disclosure may be applied to flexible endoscope systems for inspection and microsurgery systems.

### 2. EXAMINATION WITH RESPECT TO OBSERVATION USING SPECKLES

With respect to an example of a method of observing an affected part using speckles, an overview will be described, particularly, focusing on cases in which a speckle contrast is used, and then technical problems in the observation will be described.

First, speckles will be described. In imaging techniques using optical methods, there is concern regarding reduction in detection accuracy caused by generation of various types of noise, and speckle interference is known as noise. Speckle interference is a phenomenon in which a dot pattern appears on a radiation surface in response to an uneven shape of the radiation surface. Since speckle interference acts as noise according to an observation method, there are cases in which a countermeasure for reducing the influence of speckle interference is performed. On the other hand, methods of using speckle interference for observation of affected body parts have also been proposed, and a method of using a speckle contrast is conceivable as one thereof.

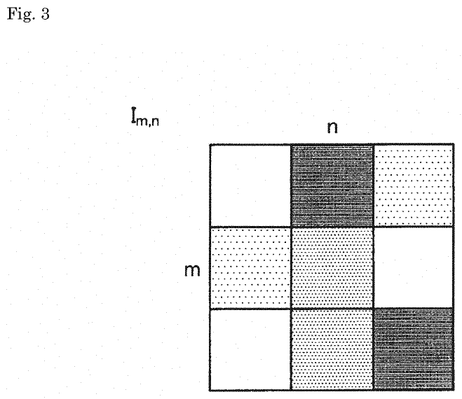

The speckle contrast is a value calculated in response to a light intensity distribution. For example, FIG. 3 is an explanatory diagram for describing an overview of speckle contrast. The speckle contrast is calculated by dividing a standard deviation of pixel values of a plurality of pixels (e.g., 3 pixels×3 pixels, 5 pixels×5 pixels, or the like) having a pixel of interest at the center by an average of the pixel values. Specifically, when a pixel value of a pixel positioned in an m-th row and an n-th column (m and n are integers equal to or greater than 1) is set to I_{m,n}, a speckle contrast is calculated for each pixel of interest through a calculation formula represented as (Formula 1) below.

$$[ Math . 1 ]$$

$$Speckle contrast = \frac{{σ}_{m , n}}{< {I}_{m , n} >} = \frac{\sqrt{< {I}_{m , n}^{2} > - < {I}_{m , n} {>}^{2}}}{< {I}_{m , n} >} \qquad ( Formula 1 )$$

In the above (Formula 1), o_{m,n} represents the standard deviation of pixel values of a plurality of pixels including a pixel positioned in the m-th row and the n-th column at the center. Further, <I_{m,n}> represents the average of the pixel values of the plurality of pixels including a pixel positioned in the m-th row and the n-th column at the center.

Here, the basic principle of the technology for allowing observation of a body part with a motion by calculating a speckle contrast is described. In a body part without a motion, a speckle contrast becomes higher because change in a speckle pattern is insignificant (ideally no change) and a standard deviation of light intensity distributions increases. In contrast, in a body part with a motion, since a speckle pattern changes in response to the motion and a relatively long exposure time (e.g., an exposure time equal to or longer than a period in which change in a motion of a target within an angle of view can be checked) is set for capturing of an image of the target, speckle patterns imaged within the exposure time are averaged and thus a speckle contrast decreases.

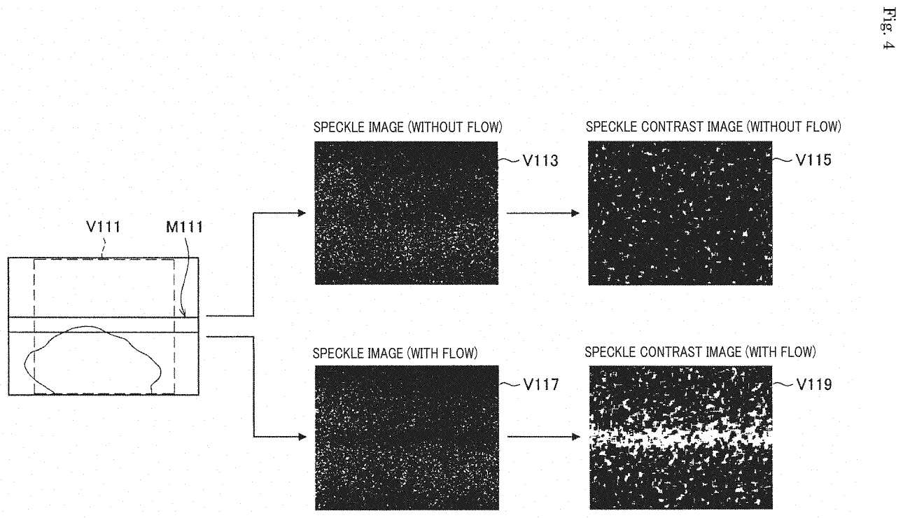

For example, FIG. 4 is an explanatory diagram for describing an overview of speckle contrast and schematically illustrates images in which speckles have been generated with respect to a target with a motion and a target without a motion (i.e., images having apparent speckles) and images based on speckle contrasts calculated with respect to each pixel of the images. Meanwhile, an image in which speckles have been generated will be referred to as a “speckle image” in the following description for convenience. In addition, an image obtained by calculating a speckle contrast with respect to each pixel of a speckle image will be referred to as a “speckle contrast image.”

Specifically, FIG. 4 illustrates speckle images and speckle contrast images in a case in which a fluid imitating blood is flowing through a flow channel M111 imitating a blood vessel and a case in which the fluid is not flowing. In FIG. 4, a reference sign V111 represents an area that is an imaging target of a speckle image. Further, a reference sign V113 represents an example of a speckle image captured in a case in which the fluid is not flowing through the flow channel M111 (i.e., a case in which there is no flow). In contrast, a reference sign V117 represents an example of a speckle image captured in a case in which the fluid is flowing through the flow channel M111 (i.e., a case in which there is a flow). As illustrated in FIG. 4, it is ascertained that speckle distributions are different between a part corresponding to the flow channel M111 having a flow and other parts (i.e., body parts other than the flow channel M111 having no flow) in the speckle image V117, as compared to the speckle image V113.

In addition, a reference sign V115 represents a speckle contrast image generated by calculating a speckle contrast with respect to each pixel of the speckle image V113. Likewise, a reference sign V119 represents a speckle contrast image generated by calculating a speckle contrast with respect to each pixel of the speckle image V117. It is ascertained from a comparison between the speckle contrast images V115 and V119 that distributions of speckle contrast calculation results are different in a body part (i.e., a body part with a motion) corresponding to the flow channel M111 and other body parts (i.e., body parts without a motion) in the speckle contrast image V119 in a case where the fluid is flowing through the flow channel M111. From these characteristics, it is possible to obtain an image presenting a blood flow by generating a speckle contrast image on the basis of imaging results of a speckle image, for example, in a case where the blood vessel is used as an observation target.

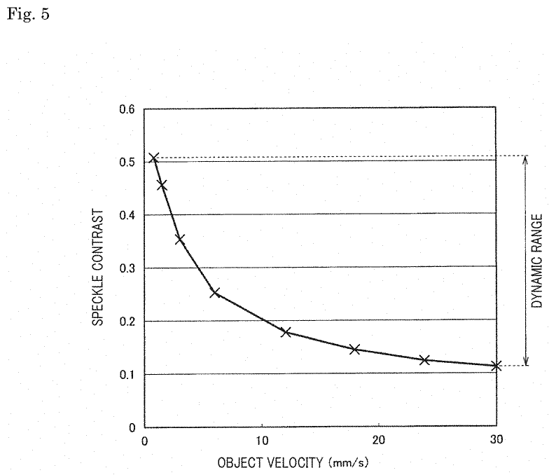

Further, FIG. 5 is an explanatory diagram for describing an example of a relationship between a speckle contrast and a motion of an object. In FIG. 5, the horizontal axis represents a velocity (mm/s) of an object (i.e., an object representing a motion) that is a target. In addition, the vertical axis represents a speckle contrast. As illustrated in FIG. 4, a speckle contrast calculation result increases as the object velocity decreases, and the speckle contrast tends to decrease according to increases in the object velocity. Meanwhile, a range of speckle contrast values that may be acquired as illustrated in FIG. 4 will be referred to as a “dynamic range” in the following description for convenience.

By using characteristics as illustrated in FIG. 5, the velocity of a motion (e.g., a blood flow) of an object that is a target may also be calculated on the basis of a speckle contrast calculation result, for example.

On the other hand, when a motion of an object (e.g., an affected body part that is an observation target) is insignificant, there are cases in which it is difficult to detect the object or the motion of the object because change in a speckle contrast associated with the motion decreases. As a solution to such a problem, a method of separating light from the object that is the observation target (e.g., light reflected from the object) into a plurality of polarized lights having different polarization directions and using only any one polarized light as an observation target (i.e., imaging target) is conceivable.

In a case where an object that is an observation target is imaged, light reflected from the object may have two orthogonal polarized light components in general. Although a speckle itself is a phenomenon occurring due to inference of light, two orthogonal polarized lights do not interfere each other so that light intensities thereof simply overlap, and as a result, speckle patterns are averaged. According to this characteristic, there are cases in which a higher speckle contrast may be obtained by observing only one of two orthogonal polarized lights.

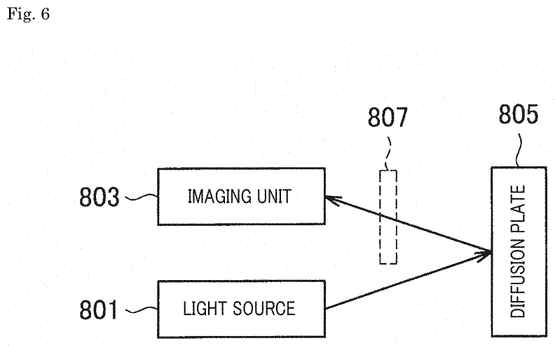

Here, an overview with respect to an example in a case where only one of two orthogonal polarized lights is an observation target when observation is performed on the basis of speckle contrast calculation results is described with reference to FIG. 6 to FIG. 8.

For example, FIG. 6 is an explanatory diagram for describing an influence on speckle contrast calculation results in a case where polarized light is used and schematically illustrates a configuration pertaining to capturing of a speckle image. Specifically, in the example illustrated in FIG. 6, a speckle image is acquired by imaging reflected light with a speckle pattern generated by reflecting light projected from a light source 801 by a diffusion plate 805 using the imaging unit 803. On the basis of this configuration, it is possible to cause an imaging unit 803 to image only one of two orthogonal polarized lights included in the reflected light from the diffusion plate 805 by interposing a polarization filter 807 between the diffusion plate 805 and the imaging unit 803, for example.

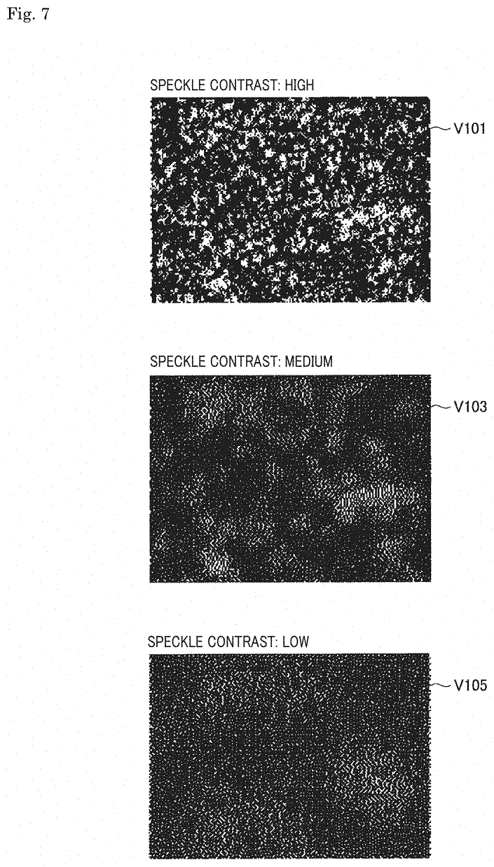

Further, FIG. 7 illustrates examples of images having different speckle contrast levels. Specifically, an image V101 has a highest speckle contrast and images V101, V103 and V105 have sequentially decreasing speckle contrasts in the example illustrated in FIG. 7.

Here, it is assumed that the image V105 illustrated in FIG. 7 is acquired as a speckle contrast image using an imaging result obtained without the polarization filter 807 in the example illustrated in FIG. 6. In this case, it is possible to acquire a speckle image with a higher speckle contrast, such as the images V103 and V101 illustrated in FIG. 7, as a speckle contrast image, for example, by interposing the polarization filter 807 in the example illustrated in FIG. 6.

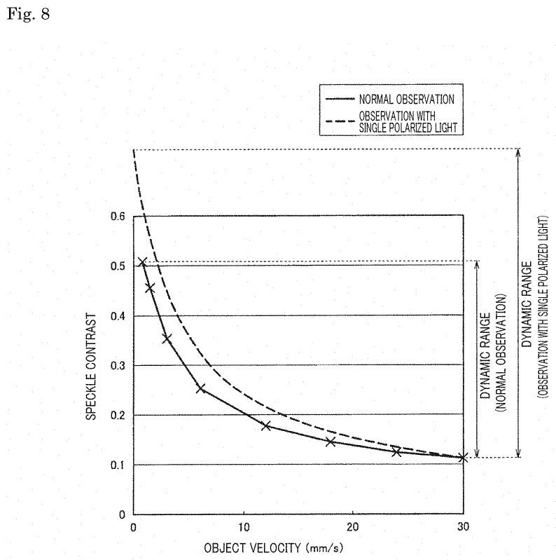

Further, FIG. 8 is an explanatory diagram for describing another example of a relationship between a speckle contrast and a motion of an object and illustrates an example when polarized light is used for observation of the object (i.e., acquisition of a speckle image). Specifically, FIG. 8 illustrates results of observation using only one of two orthogonal polarized lights in addition to the example illustrated in FIG. 5. In FIG. 8, an example represented as normal observation is illustrated as the example illustrated in FIG. 5, that is, an example of characteristics in a case where light from an object that is a target (e.g., reflected light reflected from the object) is observed without being separated into polarized light. In addition, an example represented as observation with a single polarized light is illustrated as an example of characteristics in a case where only one of two orthogonal polarized lights constituting light from the object that is the target is observed. Meanwhile, it is assumed that “normal observation” represents a case in which light from an object that is a target is observed without being separated into polarized lights in the following description for convenience unless particularly mentioned. Further, it is assumed that “observation with a single polarized light” represents a case in which only one of a plurality of polarized lights having different polarization directions (e.g., two orthogonal polarized lights) which are included in light from an object that is a target is observed unless particularly mentioned.

As illustrated in FIG. 8, in observation with a single polarized light, a speckle contrast value tends to become higher than in normal observation in a state in which the motion of the object is small (furthermore, a state in which the object stops). On the other hand, under conditions in which the motion of the object is relatively fast and the speckle contrast value becomes lower, a speckle contrast value difference between normal observation and observation with a single polarized light tends to decrease (furthermore, the difference tends to disappear). According to this characteristic, change in the speckle contrast value with respect to change in the object velocity increases (i.e., the dynamic range becomes wide) in observation with a single polarized light as compared to normal observation. Accordingly, it is possible to perform observation of the object (e.g., measurement of the object velocity) with higher sensitivity than that in normal observation by applying observation with a single polarized light even when change in the object velocity is insignificant as compared to a case in which normal observation is applied.

However, in a case where observation with a single polarized light is applied, the quantity of light available for observation decreases as compared to normal observation due to the characteristic that only one of a plurality of polarized lights having different polarization directions included in light from an object that is a target (e.g., reflected light from the object) is used for observation. That is, in a case where the quantity of light that is an observation target is insignificant, the quantity of light further decreases and a case in which it is difficult to observe the observation target may also be conceived.

In view of the aforementioned circumstances, the present disclosure proposes a technology for allowing realization of observation of an affected part with a motion in a more suitable state. As a specific example, the present disclosure proposes a technology for allowing observation of an object with higher sensitivity (e.g., realization of a wider dynamic range) and efficient utilization of light from the object (e.g., curbing of decrease in the quantity of light available for observation) to be compatible in a more suitable state.

### 3. TECHNICAL FEATURES

Hereinafter, technical features of the medical observation system according to an embodiment of the present disclosure will be described.

### 3.1. Basic Concept

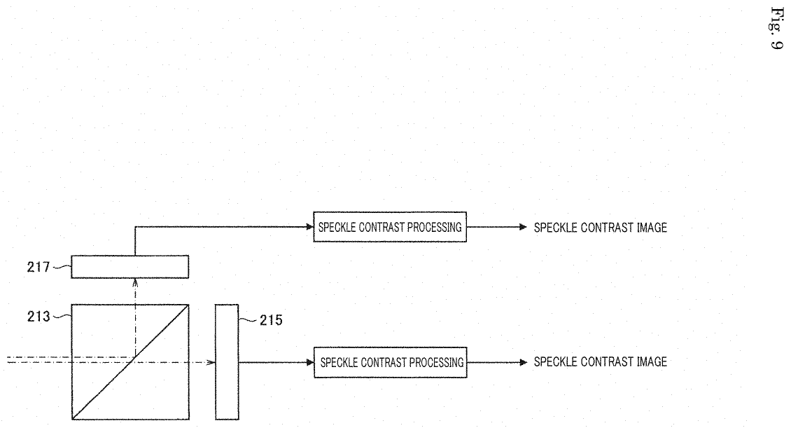

First, the basic concept of the technology pertaining to observation of an affected body part using speckles in the medical observation system according to an embodiment of the present disclosure will be described with reference to FIG. 9. FIG. 9 is an explanatory diagram for describing the basic concept of the technology pertaining to observation of an affected body part in the medical observation system according to an embodiment of the present disclosure.

In FIG. 9, a reference sign 213 represents a branching optical system that separates incident light into a plurality of polarized lights having different polarization directions. The branching optical system 213 may include, for example, a polarizing beam splitter (PBS). The branching optical system 213 may, for example, separate a plurality of polarized lights (e.g., p waves and s waves) included in incident light by reflecting some polarized lights and transmitting other polarized lights. Further, reference signs 215 and 217 schematically represent imaging elements.

That is, in the medical observation system according to the present embodiment, light from an object that is a target (e.g., reflected light reflected from the object, or the like) is separated by the branching optical system 213 into a plurality of polarized lights having different polarization directions (e.g., two polarized lights in differently orthogonal polarization directions) and the separated polarized lights are individually detected by the imaging elements 215 and 217. For example, in the example of FIG. 9, the imaging element 215 detects polarized light that has passed through the branching optical system 213 from among a plurality of polarized lights separated by the branching optical system 213 and the imaging element 217 detects a polarized light reflected by the branching optical system 213.

On the basis of the above-described configuration, the medical observation system according to the present embodiment executes processing with respect to observation of an object that is a target (e.g., an affected body part) using at least any of images (i.e., images obtained from imaging results of respective polarized lights) individually captured by the imaging elements 215 and 217. Here, the medical observation system may individually apply predetermined arithmetic operation processing to the images captured by the imaging elements 215 and 217 and execute processing with respect to observation of the object that is the target using at least any of results of application of the arithmetic operation processing to the images.

As a specific example, the medical observation system generates a speckle contrast image by calculating a speckle contrast with respect to each pixel of images (speckle images) according to imaging results of the imaging elements 215 and 217 in the example illustrated in FIG. 9. Then, the medical observation system executes processing with respect to observation of the object (e.g., affected body part) on the basis of at least any of speckle contrast images generated with respect to the plurality of polarized lights separated from light from the object.

For example, the medical observation system may combine the speckle contrast images generated with respect to the plurality of polarized lights. In this case, the medical observation system may combine the speckle contrast images generated for respective polarized lights by averaging pixel values for each pixel between the speckle contrast images generated with respect to the plurality of polarized lights, for example. In addition, as another example, the medical observation system may combine the speckle contrast images generated with respect to the plurality of polarized lights on the basis of weights in response to light intensities of the plurality of polarized lights. In this case, when the medical observation system averages the pixel values for each pixel between the speckle contrast images generated with respect to the plurality of polarized lights, the medical observation system may perform weighted averaging in which weights in response to the light intensities of the plurality of polarized lights are reflected. Meanwhile, the above-described speckle contrast image combination method is merely an example and the method is not particularly limited as long as it can combine speckle contrast images generated with respect to the plurality of polarized lights. According to this configuration, it is possible to use condensed light (in other words, light from an object) with high efficiency and obtain brighter images.

In addition, the dynamic range tends to be wider in observation with a single polarized light as compared to normal observation, as described above. That is, each speckle contrast image generated for each polarized light has a wider dynamic range than that of a speckle contrast image generated in normal observation. That is, it is possible to obtain a speckle contrast image with a wider dynamic range than that in the case of normal observation by combining speckle contrast images generated for respective polarized lights while maintaining the same degree of brightness as that in the case of normal observation.

Further, it is possible to more curb noise as compared to the case of normal observation by combining speckle contrast images generated for respective polarized lights. Specifically, general speckle image processing is performed using an average, a standard deviation or the like of pixel values in a micro area (e.g., 5×5 pixels or 7×7 pixels). However, an evaluation value in the micro area tends to easily vary according to how a speckle pattern is included in the area due to the characteristic of the evaluation value in the micro area. That is, an evaluation result in the micro area tends to significantly vary as a whole and appear to have apparent noise. On the other hand, a method of widening the micro area and increasing the number of sample pixels is conceivable, but an average value in a wide area is obtained through this method and thus there are cases in which it is difficult to obtain resolution of images after processing.

On the other hand, the technology according to an embodiment of the present disclosure can reduce noise in an evaluation image after analysis by performing evaluation (calculation) of a speckle contrast with respect to each of a plurality of polarized lights for each micro area and averaging evaluation results. That is, it is possible to substantially perform evaluation with a number of sample pixels twice that in the case of normal observation by separating condensed light (e.g., light from the affected body part) into two polarized lights, as illustrated in FIG. 9, and evaluating (calculating) a speckle contrast with respect to each polarized light.

In addition, in the medical observation system according to an embodiment of the present disclosure, it is possible to individually obtain information such as a speckle contrast with respect to each of a plurality of polarized lights separated from condensed light (e.g., light from the affected body part), as illustrated in FIG. 9. Accordingly, various analyses using these characteristics may be performed in the medical observation system according to an embodiment of the present disclosure.

As a specific example, an object (an affected body part) that is a target may be illuminated with a specific polarized light by using a laser light as a light source. In this case, light reflected from the surface of the object also has the specific polarized light component. In such a situation, there are cases in which light reflected from the surface of the object is directly observed and thus stronger light (e.g., brighter light) is observed as compared to a case in which scattered light is observed. That is, there are cases in which, in an observed image, an image signal (in other words, pixel values) according to an imaging result is saturated in a part having a strong influence of surface reflection.

On the other hand, in the technology according to an embodiment of the present disclosure, a speckle image with respect to each of a plurality of polarized lights having different polarization directions is acquired as described above. According to this characteristic, even in a case in which some speckle images corresponding to some polarized lights are saturated, for example, the influence of surface reflection may be further reduced in processing with respect to observation of an affected body part, such as signal processing in a subsequent stage, by using speckle images corresponding to other polarized lights. This is not limited to speckle images and also applies to speckle contrast images in the same manner.

The aforementioned method is merely an example and the method of using a speckle image acquired with respect to each of a plurality of polarized lights having different polarization directions and a speckle contrast image generated on the basis of a speckle image for each polarized light is not particularly limited. For example, any of a speckle image acquired for each polarized light and a speckle contrast image generated for each polarized light may be selected and used according to predetermined conditions. Further, the aforementioned speckle images and the aforementioned speckle contrast images may be combined between a plurality of polarized lights and a combined result may be used. In this manner, various types of processing with respect to observation of an affected part may be realized using at least any of a speckle image acquired for each polarized light and a speckle contrast image generated for each polarized light in the medical observation system according to the present disclosure.

The basic concept of the technology with respect to observation of an affected body part using speckles in the medical observation system according to an embodiment of the present disclosure has been described above with reference to FIG. 9.

### 3.2. Configuration Example of System

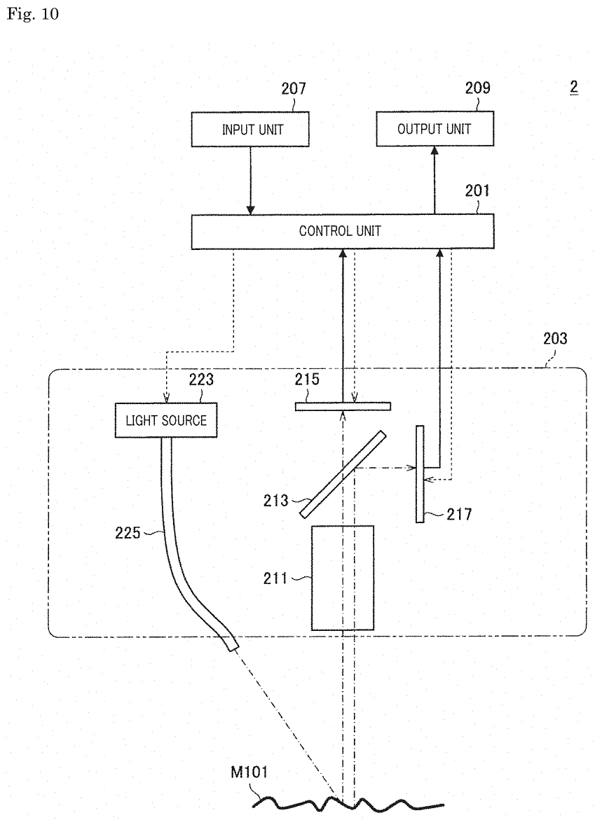

Subsequently, an example of a configuration of a medical observation system according to an embodiment of the present disclosure will be described. For example, FIG. 10 is an explanatory diagram for describing an example of a configuration of a medical observation system according to an embodiment of the present disclosure. Specifically, FIG. 10 illustrates an example of a schematic system configuration of the medical observation system in a case in which observation of an affected body part is performed on the basis of speckle images acquired by radiating light with a predetermined wavelength (e.g., a narrow-band light) to the affected body part and imaging light from the affected body part (e.g., reflected light reflected from the affected body part). Meanwhile, the medical observation system illustrated in FIG. 10 will be referred to as a “medical observation system 2” in the following description for convenience.

The medical observation system 2 includes a control unit 201, an imaging unit 203, an input unit 207, and an output unit 209 in the example illustrated in FIG. 5. The input unit 207 and the output unit 209 correspond to the input device 147 and the display device 141 in the example illustrated in FIG. 1.

The imaging unit 203 may include, for example, an imaging optical system 211, a branching optical system 213, imaging elements 215 and 217, and a light source 223.

The light source 223 corresponds to an example of the light source device 143 in the example illustrated in FIG. 1. Light projected from the light source 223 is transmitted through a transmission cable 225 configured to be able to guide light using an optical fiber or the like and radiated to an affected body part M101. Meanwhile, the wavelength of light projected from the light source 223 may be controlled or the light source 223 itself may be selectively switched in response to an observation target or an observation method. As a specific example, in the case of observation of a bright field image of an affected body part, a light source configured to be able to radiate visible light (e.g., RGB lights) may be applied as the light source 223. As another example, in the case of fluorescence observation, a light source configured to be able to project a wavelength for excitation of a fluorescent material to be used may be applied as the light source 223. As a more specific example, in a case where fluorescence observation using a fluorescent material exciting by near-infrared rays, such as indocyanine green (ICG), is performed, a light source configured to be able to radiate the near-infrared rays may be applied as the light source 223.

The branching optical system 213 and the imaging elements 215 and 217 correspond to the branching optical system 213 and the imaging elements 215 and 217 described with reference to FIG. 9. That is, the branching optical system 213 separates light incident to the imaging unit 203 (which corresponds to light from the affected body part, for example, and is simply referred to as an “incident light” hereinafter) into a plurality of polarized lights having different polarization directions, guides some separated polarized lights to the imaging element 215 and guides other polarized lights to the imaging element 217.

The imaging elements 215 and 217 are provided behind the branching optical system 213 and individually detect polarized lights separated from the incident light by the branching optical system 213. For example, imaging elements such as CCDs or CMOSs may be applied as the imaging elements 215 and 217.

The control unit 201 corresponds to the CCU 139 illustrated in FIG. 1 and controls the operation of each component of the medical observation system 2. For example, the control unit 201 may control the operation of the light source 223 in response to an observation target or an observation method. Further, the control unit 201 may control an operation with respect to capturing of an image by at least any of the imaging elements 215 and 217. Here, the control unit 201 may control image capturing conditions (e.g., a shutter speed, an aperture, a gain, etc.). In addition, the control unit 201 may acquire an image according to an imaging result of at least any of the imaging elements 215 and 217 and cause the output unit 209 to present the image. Further, at this time, the control unit 201 may perform predetermined image processing on the acquired image. Further, the control unit 201 may control the operation of each part in response to detection results of various states. As a specific example, the control unit 201 may correct blurring (e.g., hand trembling) appearing in imaging results of the imaging elements 215 and 217 in response to detection results of a motion of the imaging unit 203 obtained by various sensors (illustration thereof is omitted). Further, the control unit 201 may execute the above-described various types of processing in response to instructions from a user input through the input unit 207.

Meanwhile, the example described with reference to FIG. 10 is merely an example and does not necessarily limit the configuration of the medical observation system according to an embodiment of the present disclosure. That is, some components may be appropriately changed in response to an observation target or an observation method without departing from the above-described basic concept of the medical observation system according to the present embodiment.

An example of the configuration of the medical observation system according to an embodiment of the present disclosure has been described above with reference to FIG. 10.

### 3.3 Functional Configuration

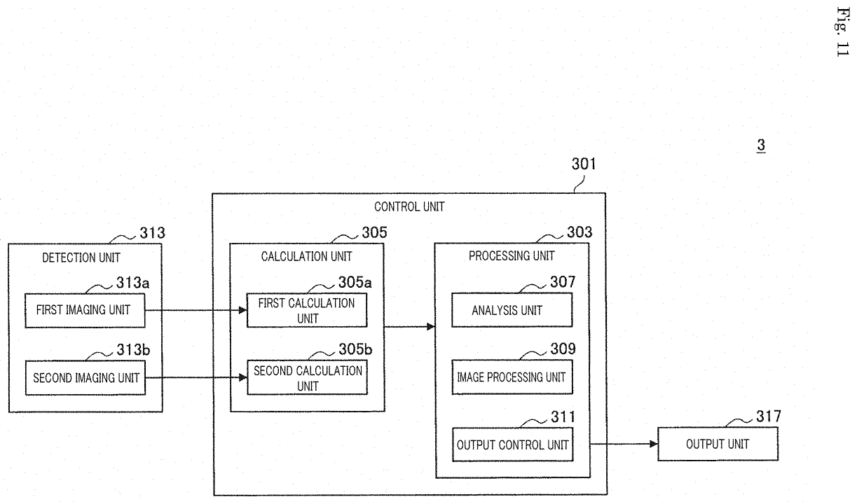

Subsequently, an example of a functional configuration of a medical observation system according to an embodiment of the present disclosure will be described particularly focusing on an example of a functional configuration of a control unit that controls the operation of each component of the medical observation system. For example, FIG. 11 is a block diagram illustrating an example of a functional configuration of a medical observation system according to an embodiment of the present disclosure. Specifically, FIG. 11 illustrates a configuration of the medical observation system according to the present embodiment particularly focusing on parts that execute various types of processing with respect to observation of an affected body part on the basis of speckle images according to detection results of a plurality of polarized lights separated from light from the affected body part. Meanwhile, the medical observation system illustrated in FIG. 11 will be referred to as a “medical observation system 3” in the following description for convenience.

As illustrated in FIG. 11, the medical observation system 3 includes a control unit 301, a detection unit 313, and an output unit 317. The output unit 317 may correspond to the output unit 209 illustrated in FIG. 10. Accordingly, detailed description of the output unit 317 is omitted.

The detection unit 313 includes a first imaging unit 313a and a second imaging unit 313b. The detection unit 313 may correspond to the imaging unit 203 illustrated in FIG. 10, for example. One of the first imaging unit 313a and the second imaging unit 313b may correspond to the imaging element 215 illustrated in FIG. 5 and the other may correspond to the imaging element 217 illustrated in FIG. 5. That is, some of a plurality of polarized lights having different polarization directions separated from light from an affected body part by the branching optical system 213 or the like illustrated in FIG. 10 are imaged (detected) by the first imaging unit 313a and other polarized lights are imaged (detected) by the second imaging unit 313b. Meanwhile, since substantially the same configuration as the imaging elements 215 and 217 illustrated in FIG. 5 may be applied to the first imaging unit 313a and the second imaging unit 313b, as described above, detailed description is omitted. Each of the first imaging unit 313a and the second imaging unit 313b outputs an image (e.g., a speckle image) according to an imaging result of a corresponding polarized light to the control unit 301.

The control unit 301 may correspond to the control unit 201 illustrated in FIG. 10. As illustrated in FIG. 11, the control unit 301 includes an arithmetic operation unit 305 and a processing unit 303.

The arithmetic operation unit 305 executes various types of arithmetic operation processing on the basis of polarized light imaging results (detection results) of the first imaging unit 313a and the second imaging unit 313b. For example, in the example illustrated in FIG. 11, the arithmetic operation unit 305 includes a first arithmetic operation unit 305a and a second arithmetic operation unit 305b. The first arithmetic operation unit 305a executes various types of arithmetic operation processing on the basis of polarized light imaging results of the first imaging unit 313a. In addition, the second arithmetic operation unit 305b executes various types of arithmetic operation processing on the basis of polarized light imaging results of the second imaging unit 313b. The first arithmetic operation unit 305a and the second arithmetic operation unit 305b may be provided as independent hardware components. Further, the first arithmetic operation unit 305a and the second arithmetic operation unit 305b may be realized as software, such as processes that individually execute processing.

For example, processing with respect to generation of a speckle contrast image may be conceived as arithmetic operation processing executed by the first arithmetic operation unit 305a and the second arithmetic operation unit 305b. For example, the first arithmetic operation unit 305a may calculate a speckle contrast using each pixel of an image (speckle image) acquired according to a polarized light imaging result of the first imaging unit 313a as a pixel of interest and generate a speckle contrast image on the basis of the calculation result. Likewise, the second arithmetic operation unit 305b may generate a speckle contrast image on the basis of an image acquired according to a polarized light imaging result of the second imaging unit 313b.

Of course, the aforementioned processing is merely an example and does not necessarily limit details of arithmetic operation processing executed by the first arithmetic operation unit 305a and the second arithmetic operation unit 305b. That is, the first arithmetic operation unit 305a and the second arithmetic operation unit 305b may appropriately change arithmetic operation processing to be applied to polarized light imaging results in response to processing with respect to observation of an affected body part executed in a subsequent stage. For example, as an example of blood flow observation, a method of using light Doppler, that is, a method of calculating a blood flow velocity by catching an optical frequency shift occurring when light is scattered by a blood flow may be conceived. In this case, the first arithmetic operation unit 305a and the second arithmetic operation unit 305b may execute processing with respect to detection (extraction) of an optical frequency shift on the basis of imaging results of polarized lights corresponding thereto.

Then, the arithmetic operation unit 305 outputs the aforementioned arithmetic operation results for each polarized light, obtained by the first arithmetic operation unit 305a and the second arithmetic operation unit 305b, to the processing unit 303. Meanwhile, to make features of the medical observation system 3 be ascertained more easily, the following description focuses on a case in which the arithmetic operation unit 305 outputs speckle contrast images individually generated for each polarized light by the first arithmetic operation unit 305a and the second arithmetic operation unit 305b to the processing unit 303. In this case, the arithmetic operation unit 305 may output speckle images (i.e., images according to imaging results of polarized lights) that are speckle contrast image generation sources to the processing unit 303.

The processing unit 303 acquires, from the arithmetic operation unit 305, the arithmetic operation results individually applied to the plurality of polarized lights having different polarization directions separated from the light from the affected body part and executes processing with respect to observation of the affected body part in response to at least any of the arithmetic operation results for each polarized light. For example, the processing unit 303 may acquire, from the arithmetic operation unit 305, speckle contrast images individually generated with respect to the plurality of polarized lights having different polarization directions separated from the light from the affected body part. The processing unit 303 executes processing with respect to observation of the affected body part on the basis of at least any of the speckle contrast images individually generated with respect to the plurality of polarized lights. As a specific configuration example assuming this case (i.e., an example of a configuration in which processing with respect to observation of the affected body part is executed), the processing unit 303 includes an analysis unit 307, an image processing unit 309, and an output control unit 311 in the example illustrated in FIG. 3.

The analysis unit 307 executes various types of analysis processing on the basis of the acquired speckle contrast images. As a specific example, the analysis unit 307 may calculate a moving velocity of an object (in other words, the affected body part that is an observation target) included in at least some area in the acquired speckle contrast images on the basis of pixel values of pixels included in the area (i.e., speckle contrast calculation values).

In addition, the analysis unit 307 may extract a characteristic part (e.g., a part corresponding to the affected body part) from the speckle contrast images by performing image analysis on the speckle contrast images.

Further, the analysis unit 307 may perform predetermined determination on the basis of results of image analysis by performing the image analysis on the speckle contrast images. As a specific example, the analysis unit 307 may determine whether at least some of the speckle contrast images are saturated by evaluating a pixel value of each pixel of the speckle contrast images. By using this determination result, for example, when saturation occurs in speckle contrast images corresponding to some polarized lights from among the speckle contrast images corresponding to the plurality of polarized lights, it is possible to select speckle contrast images corresponding to other polarized lights as a target of subsequent processing.

Meanwhile, the analysis unit 307 may use only a speckle contrast image corresponding to any of the plurality of polarized lights as an analysis target. As another example, the analysis unit 307 may use the speckle contrast images corresponding to the plurality of polarized lights as an analysis target. Further, as another example, the analysis unit 307 may use an image obtained by combining the speckle contrast images corresponding to the plurality of polarized lights as an analysis target. Meanwhile, the combination may be executed, for example, by the image processing unit 309 which will be described later.

The image processing unit 309 performs various types of image processing on the acquired speckle contrast images. For example, the image processing unit 309 may execute processing with respect to adjustment of brightness, contrast, tone, and the like on the acquired speckle contrast images.

In addition, the image processing unit 309 may combine the speckle contrast images corresponding to the plurality of polarized lights. As a specific example, the image processing unit 309 may combine the speckle contrast images corresponding to the plurality of polarized lights by averaging pixel values for each pixel between the speckle contrast images corresponding to the plurality of polarized lights.

Meanwhile, the aforementioned analysis unit 307 and image processing unit 309 are not limited to only the speckle contrast images and may use speckle images that are generation sources of the speckle contrast images as the aforementioned various types of processing target.

The output control unit 311 causes the output unit 317 to output various types of information as display information to present the information. For example, the output control unit 311 may cause the output unit 317 to output, as display information, a speckle contrast image generated for each polarized light or a speckle image that is a generation source of the speckle contrast image. In addition, the output control unit 311 may cause the output unit 317 to output, as display information, an image obtained by the image processing unit 309 combining the speckle contrast images corresponding to the plurality of polarized lights or an image obtained by combining the speckle images that are generation sources of the speckle contrast images. Further, the output control unit 311 may cause the output unit 317 to output information according to an analysis result of the analysis unit 307 (e.g., a velocity of the object that is an observation target). In addition, the output control unit 311 may control information caused to be output from the output unit 317 in response to a determination result of the analysis unit 307.

Further, the output control unit 311 may associate two or more of the above-described various types of information and cause the output unit 317 to output the associated information. As a specific example, the output control unit 311 may cause the output unit 317 to output display information in which information according to a calculation result of the velocity of the object calculated on the basis of a speckle contrast image is superposed on the speckle contrast image. Further, the output control unit 311 may cause the output unit 317 to output display information in which two or more images of speckle contrast images for respective polarized lights and speckle images for respective polarized lights are associated and presented. As a specific example, the output control unit 311 may cause the output unit 317 to output display information in which the two or more images are arranged and presented. Further, as another example, the output control unit 311 may cause the output unit 317 to output, as display information, a so-called picture-in-picture (PIP) image in which, on an area of an image, another image is superposed. In addition, the output control unit 311 may selectively switch information caused to be output from the output unit 317 as display information according to predetermined conditions.

Meanwhile, the above-described functional configuration is merely an example and the functional configuration of the medical observation system is not necessarily limited to the example illustrated in FIG. 11 if it can realize the aforementioned operation of each component. As a specific example, at least any of the detection unit 313 and the output unit 317 and the control unit 301 may be integrated. Further, as another example, some functions of the control unit 301 may be provided outside the control unit 301. In addition, at least some functions of the control unit 301 may be realized by a plurality of devices operating in cooperation. Further, some components of the medical observation system may be changed or other components may be further added without departing from the above-described technical features of the medical observation system according to the present embodiment.

Meanwhile, an apparatus including components corresponding to the control unit 301 illustrated in FIG. 11 corresponds to an example of the “medical observation apparatus.”

An example of the functional configuration of the medical observation system according to an embodiment of the present disclosure has been described above particularly focusing on an example of the functional configuration of the control unit that control the operation of each component of the medical observation system with reference to FIG. 11.

### 3.4. Processing

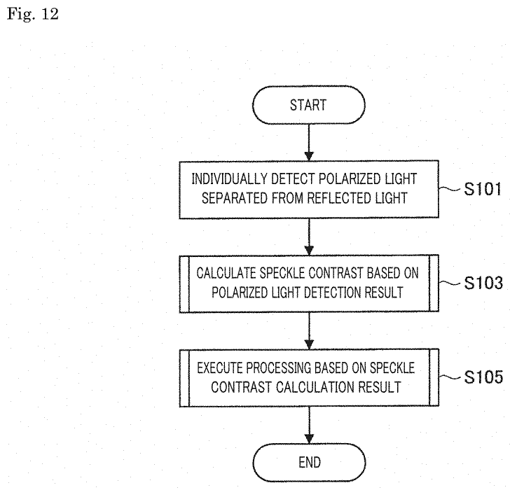

Subsequently, an example of a flow of a series of processes of the medical observation system according to an embodiment of the present disclosure will be described particularly focusing on the operation of the control unit 301 illustrated in FIG. 11. For example, FIG. 12 is a flowchart illustrating an example of a flow of a series of processes of the medical observation system according to an embodiment of the present disclosure.

First, the detection unit 313 individually detects (images) a plurality of polarized lights having different polarization directions separated from light from an affected body part by the branching optical system 213 illustrated in FIG. 10, or the like. The detection unit 313 individually outputs images (speckle images) according to detection results of the plurality of polarized lights to the control unit 301 (S101).

The control unit 301 (arithmetic operation unit 305) individually acquires the images according to the detection results of the plurality of polarized lights from the detection unit 313. The control unit 301 (arithmetic operation unit 305) individually applies predetermined arithmetic operation processing to the detection results of the plurality of polarized lights. As a specific example, the control unit 301 (arithmetic operation unit 305) calculates a speckle contrast using each pixel of the images (speckle images) according to the detection results of the plurality of polarized lights as a pixel of interest and generates speckle contrast images on the basis of results of the calculation with respect to the plurality of polarized lights (S103).

Subsequently, the control unit 301 (processing unit 303) executes processing with respect to observation of the affected body part according to arithmetic operation results with respect to at least some polarized lights from among the arithmetic operation results for the detection results of the plurality of polarized lights. As a specific example, the control unit 301 (processing unit 303) executes processing with respect to observation of the affected body part on the basis of at least some of speckle contrast images generated with respect to the plurality of polarized lights (S105).

As a more specific example, the control unit 301 (analysis unit 307) may calculate a moving velocity of an object included in at least an area of the speckle contrast images on the basis of pixel values of pixels included in the area. In addition, the control unit 301 (output control unit 311) may cause the output unit 317 to output, as display information, at least some of speckle contrast images generated for respective polarized lights. Further, the control unit 301 (image processing unit 309) may combine the speckle contrast images corresponding to the plurality of polarized lights by averaging pixel values for each pixel between the speckle contrast images corresponding to the plurality of polarized lights. In this case, the combined image may be used as a target of the aforementioned processing with respect to analysis or the aforementioned processing with respect to output.

An example of a flow of a series of processes of the medical observation system according to an embodiment of the present disclosure has been described above particularly focusing on the operation of the control unit 301 illustrated in FIG. 11 with reference to FIG. 12.

### 3.5. Modified Examples

Subsequently, modified examples of the medical observation system according to an embodiment of the present disclosure will be described.

### Modified Example 1: Example of Configuration for Individually Detecting Each Polarized Light

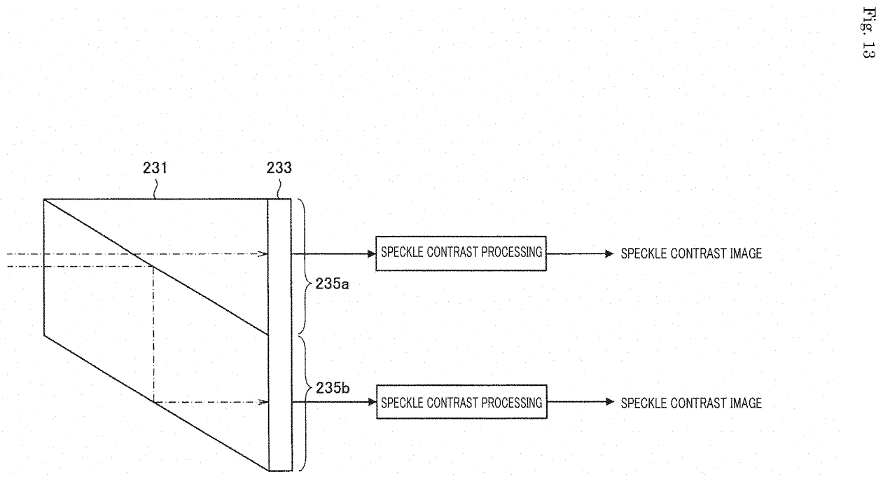

First, as modified example 1, an example of a configuration for separating light from an affected body part into a plurality of polarized lights and individually detecting (imaging) each polarized light will be described particularly focusing on a configuration corresponding to the branching optical system 213 and the imaging elements 215 and 217 in the example illustrated in FIG. 9. For example, FIG. 13 is an explanatory diagram for describing an overview of a medical observation system according to modified example 1 and illustrates an example of a configuration for separating light from an affected body part into a plurality of polarized lights and individually detecting each polarized light.

The medical observation system according to modified example 1 differs from the medical observation system according to the aforementioned embodiment (refer to FIG. 9 and FIG. 10, for example) in that light from the affected body part is separated into a plurality of polarized lights having different polarization directions and each polarized light is individually detected by a single imaging element. Specifically, in FIG. 13, a reference sign 231 represents a branching optical system that separates incident light into a plurality of polarized lights having different polarization directions and corresponds to the branching optical system 213 in the example illustrated in FIG. 9. In addition, a reference sign 233 schematically illustrates an imaging element which corresponds to the imaging elements 215 and 217 in the example illustrated in FIG. 9 and the detection unit 313 (i.e., the first imaging unit 313a and the second imaging unit 313b) in the example illustrated in FIG. 11.

That is, in the example illustrated in FIG. 13, some of the plurality of polarized lights separated from the incident light (i.e., the light from the affected body part) by the branching optical system 231 are guided to (imaged on) an area denoted by a reference sign 235a in a light-receiving surface of the imaging element 233. In addition, other polarized lights of the plurality of polarized lights are guided to (imaged on) an area denoted by a reference sign 235b in the light-receiving surface of the imaging element 233. That is, in the example illustrated in FIG. 13, images (speckle images) are individually generated on the basis of polarized light detection results (imaging results) of the areas 235a and 235b of the light-receiving surface of the imaging element 233 and speckle contrast processing is individually performed on the images. Accordingly, speckle contrast images with respect to the plurality of polarized lights separated from the incident light are individually generated.

According to the aforementioned feature, the medical observation system according to modified example 1 may capture speckle images with respect to a plurality of polarized lights using a single imaging element. Meanwhile, there are cases in which a difference is generated between light paths through which polarized lights are guided to corresponding areas of the imaging surface of the imaging element 233 between the plurality of polarized lights separated from the incident light in the example illustrated in FIG. 13. In this case, a light path with respect to at least one polarized light may be adjusted, for example, by interposing another optical system such as a relay lens.

An example of the configuration for separating light from an affected body part into a plurality of polarized lights and individually detecting (imaging) each polarized light has been described above particularly focusing on the configuration corresponding to the branching optical system 213 and the imaging elements 215 and 217 in the example illustrated in FIG. 9 as modified example 1 with reference to FIG. 13.

### Modified Example 2: Another Example of Configuration for Individually Detecting Each Polarized Light

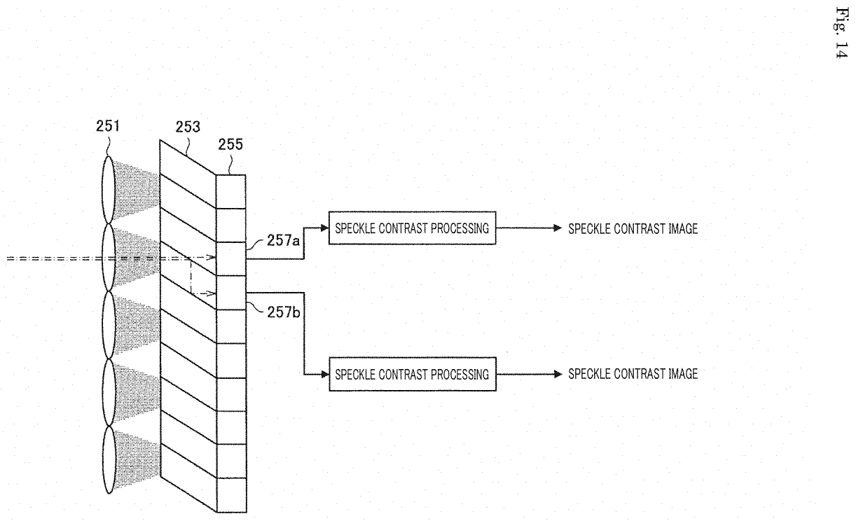

Subsequently, as modified example 2, another example of a configuration for separating light from an affected body part into a plurality of polarized lights and individually detecting (imaging) each polarized light will be described particularly focusing on a configuration corresponding to the branching optical system 213 and the imaging elements 215 and 217 in the example illustrated in FIG. 9. For example, FIG. 14 is an explanatory diagram for describing an overview of a medical observation system according to modified example 2 and illustrates an example of a configuration for separating light from an affected body part into a plurality of polarized lights and individually detecting each polarized light.

In the medical observation system according to modified example 2, light from an affected body part is separated into a plurality of polarized lights having different polarization directions and each polarized light is individually detected by a single imaging element as in the medical observation system according to modified example 1. On the other hand, in the medical observation system according to modified example 2, a light-receiving surface of an image sensor is divided into a plurality of areas in units finer than in the case of the medical observation system according to modified example 1 and any of the plurality of polarized lights separated from the incident light is guided to (imaged on) to each of the plurality of divided areas.

For example, in FIG. 14, a reference sign 253 represents polarization separation elements that separate incident light into a plurality of polarized lights having different polarization directions. That is, the configuration corresponding to the branching optical system 231 in the example illustrated in FIG. 13 includes a plurality of polarization separation elements 253 in the example illustrated in FIG. 14. The polarization separation elements 253 may be composed of a PBS, anisotropic crystals, or the like, for example. In addition, a reference sign 255 schematically represents an imaging element which corresponds to the imaging element 233 in the example illustrated in FIG. 13. That is, the imaging element 255 in the example illustrated in FIG. 14 corresponds to the imaging elements 215 and 217 in the example illustrated in FIG. 9 and the detection unit 313 (i.e., the first imaging unit 313a and the second imaging unit 313b) in the example illustrated in FIG. 11. In the example illustrated in FIG. 14, the plurality of polarization separation elements 253 separate the incident light into a plurality of polarized lights having different polarization directions. Then, the plurality of polarized lights separated from the incident light by the plurality of polarization separation elements 253 are respectively guided to (imaged on) different areas of the light-receiving surface of the imaging element 255.

According to the aforementioned characteristic, an optical system 251 for guiding some of the incident light to the polarization separation elements 253 may be provided in a previous stage of the polarization separation elements 253 in the example illustrated in FIG. 14. The optical system 251 may be configured as an array lens in which condensing lens are arrayed, for example.

According to the above-described configuration, some of the plurality of polarized lights separated from the incident light by some polarization separation elements 253 are guided to (imaged on) an area denoted by a reference sign 257a in the light-receiving surface of the imaging element 255. Other polarized lights of the plurality of polarized lights are guided to (imaged on) an area denoted by a reference sign 257b in the light-receiving surface of the imaging element 255. Meanwhile, areas to which the plurality of polarized lights separated by the polarization separation elements 253 are guided, such as the areas 257a and 257b, may be designated as areas including one or more unit areas constituting the light-receiving surface of the imaging element 255, such as lines or tiles, for example. On the basis of the above-described configuration, in an area to which polarized lights in the same polarization direction are guided from among the areas of the light-receiving surface of the imaging element 255, detection results of the polarized lights are combined to individually generate an image (speckle image) with respect to each of the plurality of polarized lights separated from the incident light. Then, speckle contrast processing is performed on the image generated for each polarized light to generate a speckle contrast image corresponding to the polarized light.

According to the aforementioned feature, the medical observation system according to modified example 2 may capture speckle images with respect to a plurality of polarized lights using a single imaging element like the medical observation system according to modified example 1. In addition, the medical observation system according to modified example 2 may reduce differences between light paths through which the plurality of polarized lights separated from the incident light by the polarization separation elements 253 are guided to corresponding areas of the imaging surface of the imaging element 255, as compared to the medical observation system according to modified example 1.

Another example of the configuration for separating light from an affected body part into a plurality of polarized lights and individually detecting (imaging) each polarized light has been described above particularly focusing on the configuration corresponding to the branching optical system 213 and the imaging elements 215 and 217 in the example illustrated in FIG. 9 as modified example 2 with reference to FIG. 14.

### Modified Example 3: Example of Method of Combining Speckle Contrast Images

Subsequently, an example of a method of combining speckle contrast images generated with respect to a plurality of polarized lights separated from light from an affected body part will be described as modified example 3.

In images in which a plurality of polarized lights have been captured (polarized light images), the strengths of surface reflection components may be inferred by comparing light intensities with respect to some common areas of the images. In an area having a significant light intensity difference between polarized light images corresponding to polarized lights, it is inferred that surface reflection of a polarized light corresponding to a polarized light image representing a larger light intensity value is dominant. Accordingly when speckle contrast images generated with respect to the plurality of polarized lights are combined, for example, weighted averaging in consideration of weights in response to light intensities of the respective polarized lights may be performed instead of simple averaging of pixel values. According to this configuration, a speckle contrast image having a smaller influence of surface reflection may also be acquired, for example.

Meanwhile, although an example of combination of speckle contrast images generated for respective polarized lights has been described above, this does not necessarily limit the configuration of medical observation system according to modified example 3. As a specific example, the medical observation system may combine speckle images according to detection results of polarized lights through the same method as that in the above-described case of combining speckle contrast images.

In particular, there are cases in which, in a brain surgery, treatment is performed while applying a physiological saline solution such that the surface is not dried. In such cases, observation is performed in a state in which the solution is present on the surface and thus surface reflection is likely to occur at the interface between the solution and the air. Even in this situation, the medical observation system according to modified example 3 may obtain an image (e.g., a speckle contrast image) in which the influence of surface reflection is further reduced.

An example of the method of combining speckle contrast images generated with respect to a plurality of polarized lights separated from light from an affected part has been described above as modified example 3.

### Modified Example 4: Example of Control in Response to Detection Result of Each Polarized Light

Subsequently, an example of control of processing executed in a subsequent stage in response to detection results of a plurality of polarized lights separated from light from an affected body part will be described as modified example 4.

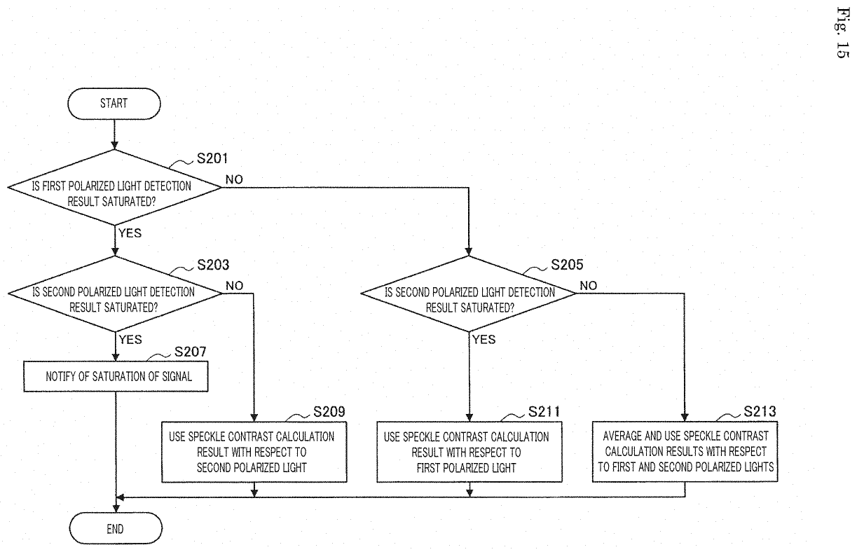

For example, FIG. 15 is an explanatory diagram for describing an example of processing of a medical observation system according to modified example 4 and represents an example of a processing flow for further reducing the influence of surface reflection. Specifically, in a situation in which an object (affected body part) that is a target is illuminated with a specific polarized light, as described above, there are cases in which an image signal (in other words, pixel values) according to an imaging result is saturated in a part having a strong influence of surface reflection. Accordingly in the example illustrated in FIG. 15, processing in a subsequent stage is selectively switched in response to whether image signals according to detection results of a plurality of polarized lights separated from the light from the affected body parts are saturated. Meanwhile, it is assumed that two orthogonal polarized lights in different polarized directions separated from the light from the affected body part are individually detected in the following description. Further, one of the two polarized lights is referred to as a “first polarized light” and the other is referred to as a “second polarized light” for convenience.

First, a case in which a detection result of the first polarized light is not saturated (NO in S201) and a detection result of the second polarized light is not saturated (NO in S205) is described. In this case, the medical observation system uses speckle contrast calculation results (e.g., speckle contrast images) with respect to the first polarized light and the second polarized light for observation of the affected body part (e.g., evaluation of blood flow motion, or the like) (S213). As a specific example, the medical observation system averages the speckle contrast calculation results with respect to the first polarized light and the second polarized light and uses the averaged speckle contrast for observation of the affected body part.

Next, an example of a case in which any of the detection results of the first polarized light and the second polarized light is saturated will be described. For example, when the detection result of the first polarized light is not saturated (NO in S201) and the detection result of the second polarized light is saturated (YES in S205), the medical observation system uses the speckle contrast calculation result with respect to the first polarized light for observation of the affected body part (S211). In addition, when the detection result of the first polarized light is saturated (YES in S201) and the detection result of the second polarized light is not saturated (NO in S203), the medical observation system uses the speckle contrast calculation result with respect to the second polarized light for observation of the affected body part (S209).

On the other hand, a case in which the detection result of the first polarized light is saturated (YES in S201) and the detection result of the second polarized light is saturated (YES in S203) may also be conceived. In this case, it is inferred that the medical observation system has difficulty using the detection results of both the first polarized light and the second polarized light for observation of the affected body part. Accordingly, the medical observation system may notify a user that the detection results of both the first polarized light and the second polarized light are saturated through the output unit, for example (S207).

An example of control of processing executed in the subsequent stage in response to detection results of a plurality of polarized lights separated from an affected body part has been described above as modified example 4 with reference to FIG. 15.

### 3.6. Operation Effects

In medical fields, blood flow observation is required for various purposes. For example, as an example in which blood observation is required, a situation in which a procedure on cerebral aneurysm is performed may be conceived. Cerebral aneurysm refers to a body part in which a part of brain blood vessels (artery) has swollen and weakened. Largely swollen cerebral aneurysm is likely to rupture and cause bleeding in the future. Accordingly, there are cases in which treatment for blocking flow of blood is performed, for example, by pinching (i.e., clipping) the neck of the aneurysm in order to preventively block flow of blood to the aneurysm. Here, blood flow observation for checking whether flow of blood to the aneurysm is blocked by clipping (i.e., presence or absence of flow of blood to the aneurysm) is performed.

In addition, checking that microvessels branching from the artery, called perforators, are not clipped is as an important point in clipping of the aneurysm. The perforators are microvessels, and when these blood vessels are clipped, it is likely to cause a significant obstacle to functions of the brain to which the blood vessels deliver oxygen and nutrients. Although the perforators are important as described above, they are blood vessels of about 1 mm or less and it is difficult to evaluate presence or absence of a blood flow through an ultrasonic Doppler blood flowmeter. On the other hand, a technology for observing a target on the basis of image capture results, such as speckle blood flow imaging, has higher resolution than the ultrasonic Doppler blood flowmeter and can check presence or absence of blood flow even in blood vessels of 1 mm or less.

In addition, as described above with reference to FIG. 8, it is possible to acquire a higher speckle contrast by performing observation on the basis of polarized lights separated from light from an observation target (affected body part) in cases where the observation target is stopped state as compared to normal observation in which separation into polarized lights is not performed. Further, even in cases where a motion of an observation target is sufficiently fast, the speckle contrast decreases to the same degree as that in normal observation. According to such characteristics, change in the speckle contrast (dynamic range) with respect to a velocity of the motion of the observation target is more increased, as compared to normal observation, by performing observation on the basis of polarized lights separated from light from the observation target (affected body part). According to such characteristics, it is possible to perform measurement with relatively high sensitivity even for insignificant velocity change, for example.

Since blood flow change in microvessels can be observed more suitably by catching insignificant velocity change in blood flow, for example, the effect of allowing occurrence of a situation in which blood flow is blocked through clipping to be prevented in advance is expected. In particular, with respect to microvessels, there are cases in which, when they are clipped by a clip once, the blocked state is not rapidly returned to the original state even when the clip is released. In view of this point, reduction of a risk of wrongly applying a clip as described above is considered to be important, and the effect of reducing the risk may be expected by applying the technology according to the present disclosure.

Furthermore, in a case where a speckle contrast is calculated on the basis of a speckle pattern, an average of luminances and a deviation of the luminances in a certain calculation area (e.g., a pixel area having a predetermined extent having a pixel of interest as a center) are generally calculated. When the calculation area increases, the resolution of acquired speckle contrast images tends to decrease and thus the size of the calculation area is limited in many cases. On the other hand, since a speckle contrast is calculated in the calculation area with a limited size, variation in calculated speckle contrast values tends to relatively increase in response to a size of pixel values included in the calculation area. A speckle contrast image acquired on the basis of such conditions appears to be a so-called image with significant noise in which luminance varies as a whole.

On the other hand, in the medical observation system according to the present disclosure, light from an observation target (affected body part) is separated into a plurality of polarized lights and a speckle pattern is individually imaged for each polarized light, as described above. Since speckle patterns formed for respective polarized lights are different in general, speckle contrast values calculated for respective polarized lights are also different. According to this characteristic, for example, luminance variation such as noise may be further reduced by combining speckle contrast images generated for respective polarized lights through averaging of pixel values of pixels (i.e., an image with further reduced noise may be acquired).

In particular, there are cases in which it is difficult to recognize microvessels in a situation in which there is luminance variation such as noise. As described above, there are parts that play a significant role, such as perforators that deliver oxygen and nutrients to each part of the brain, for example, even though they are microvessels. Accordingly, such microvessels may be recognized more clearly and thus the effect of further reducing a risk such as damaging of the microvessels may be expected.

### 3.7. Supplement

Meanwhile, although the above description focuses on methods of observing an affected body part mainly using speckle images and speckle contrast images, targets to which the medical observation system according to the present disclosure is applied are not necessarily limited thereto. That is, the medical observation system according to the present disclosure has the characteristic configuration in which light from an observation target (e.g., affected body part) is separated into a plurality of polarized lights having different polarization directions, the plurality of polarized lights are individually detected, and then processing with respect to observation of the target is executed on the basis of at least any of detection results of the plurality of polarized lights. Accordingly, the medical observation system according to the present disclosure may be applied to systems for enabling observation of a target by capturing an image of the target using imaging elements and observation methods using the systems.

For example, in the case of focusing on blood flow observation, a method of using the aforementioned light Doppler and a method of using a fluorescent agent may be conceived in addition to the method of using a speckle contrast.

As a more specific example, when the method of using the light Doppler is applied, processing with respect to extraction of optical frequency shift may be individually performed, for example, on the basis of detection results (imaging results) of a plurality of polarized lights separated from light from an observation target. Then, a velocity of the observation target (e.g., blood flow velocity) may be calculated on the basis of at least any of optical frequency shift extraction results corresponding to the plurality of polarized lights. Further, the velocity of the observation target (e.g., blood flow velocity) may be calculated by combining the frequency shift extraction results corresponding to the plurality of polarized lights and using it.

In addition, the method of using a fluorescent agent is a method of injecting a fluorescent agent such as ICG agent into blood and observing a fluorescent image. In this method, fluorescence is observed through the blood vessels in accordance with blood flow after injection of the fluorescent agent by imaging fluorescence emitted from the fluorescent agent (e.g., fluorescence excited by light from a light source) using imaging elements. Accordingly, information about blood flow may be obtained by performing temporal analysis on the basis of observation results of the fluorescence, for example. When this method is applied, the aforementioned temporal analysis may be performed using at least any of imaging results (i.e., fluorescent images) of a plurality of polarized lights separated from light from an observation target, for example. Further, the imaging results of the plurality of polarized lights may be combined on the basis of predetermined conditions and the aforementioned temporal analysis may be performed on the combination result.

Although the above description focuses on cases in which the present disclosure is applied to blood flow observation, targets to which the medical observation system is applied are not limited to blood flow observation as long as the characteristic configuration of the medical observation system according to the above-described present embodiment can be used.

### 4. EXAMPLE OF HARDWARE CONFIGURATION

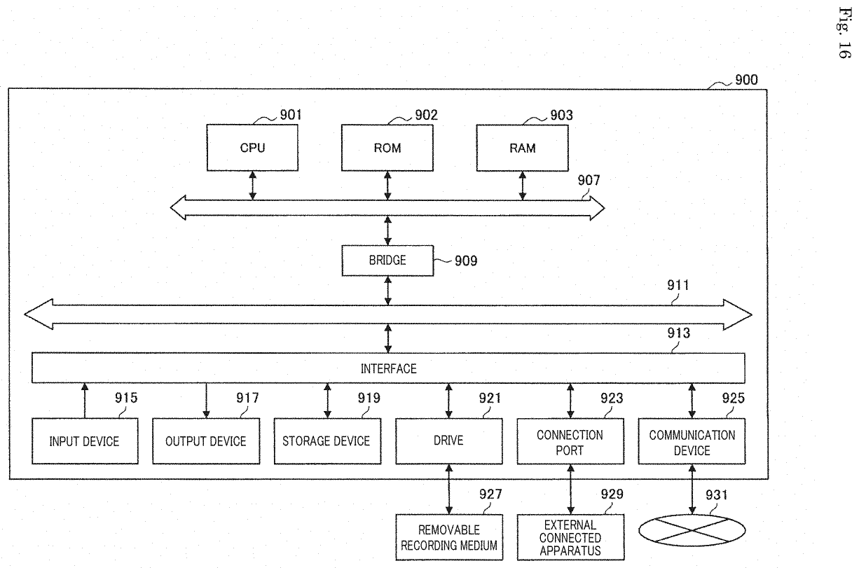

Subsequently, an example of a hardware configuration of an information processing apparatus (e.g., the control unit 201 illustrated in FIG. 10, the control unit 301 illustrated in FIG. 11, and the like) which executes various types of processing in the medical observation system according to the present embodiment will be described in detail with reference to FIG. 16. FIG. 16 is a functional block diagram illustrating a configuration example of a hardware configuration of an information processing apparatus constituting the medical observation system according to an embodiment of the present disclosure.

The information processing apparatus 900 constituting the medical observation system according to the present embodiment mainly includes a CPU 901, a ROM 902, and a RAM 903. In addition, the information processing apparatus 900 further includes a host bus 907, a bridge 909, an external bus 911, an interface 913, an input device 915, an output device 917, a storage device 919, a drive 921, a connection port 923, and a communication device 925.

The CPU 901 serves as an arithmetic operation processing device and a control device and controls all or some operations in the information processing apparatus 900 according to various programs recorded in the ROM 902, the RAM 903, the storage device 919 or a removable recording medium 927. The ROM 902 stores programs, arithmetic operation parameters and the like used by the CPU 901. The RAM 903 primarily stores programs, parameters appropriately changing in execution of programs, and the like used by the CPU 901. These are connected to each other through the host bus 907 configured as an internal bus such as a CPU bus. Meanwhile, the components of the control unit 301 illustrated in FIG. 11, that is, the arithmetic operation unit 305 (i.e., the first arithmetic operation unit 305a and the second arithmetic operation unit 305b) and the processing unit 303 (i.e., the analysis unit 307, the image processing unit 309, and the output control unit 311) may be realized by the CPU 901.

The host bus 907 is connected to the external bus 911 such as a peripheral component interconnect/interface (PCI) bus through the bridge 909. In addition, the input device 915, the output device 917, the storage device 919, the drive 921, the connection port 923, and the communication device 925 are connected to the external bus 911 through the interface 913.

The input device 915 is an operation means operated by a user, such as a mouse, a keyboard, a touch panel, a button, a switch, a lever, and a pedal, for example. In addition, the input device 915 may be, for example, a remote control means (so-called remote controller) using infrared rays or other radio waves or an external connection apparatus 929 such as a cellular phone, a PDA or the like corresponding to operation of the information processing apparatus 900. Further, the input device 915 may be composed of for example, a control circuit or the like which generates an input signal on the basis of information input by the user using the aforementioned operation means and outputs the input signal to the CPU 901. A user of the information processing apparatus 900 can input various types of data or instruct processing operation with respect to the information processing apparatus 900 by operating the input device 915.

The output device 917 is configured as a device capable of visually or acoustically notifying a user of acquired information. As such a device, there are display devices such as a CRT display device, a liquid crystal display device, a plasma display device, an EL display device and a lamp, audio output devices such as a speaker and a headphone, a printer device, and the like. The output device 917 may output, for example, results obtained by various types of processing performed by the information processing apparatus 900. Specifically, a display device displays results obtained by various types of processing performed by the information processing apparatus 900 as text or images. On the other hand, an audio output device converts an audio signal composed of reproduced voice data, audio data, and the like into an analog signal and outputs the analog signal. Meanwhile, the output unit 317 illustrated in FIG. 11 may be realized by the output device 917.

The storage device 919 is a device for data storage, which is configured as an example of a storage unit of the information processing apparatus 900. The storage device 919 may be configured as, for example, a magnetic storage disk such as a hard disk drive (HDD), a semiconductor storage device, an optical storage device, a magneto-optical storage device, or the like. The storage device 919 stores programs executed by the CPU 901, various types of data, and the like.

The drive 921 is a reader/writer for recording media and is embedded in the information processing apparatus 900 or attached to the outside thereof. The drive 921 reads information recorded in the removable recording medium 927 inserted therein, such as a magnetic disk, an optical disc, a magneto-optical disc, or a semiconductor memory and outputs the read information to the RAM 903. In addition, the drive 921 may write records in the removable recording medium 927 inserted therein, such as a magnetic disk, an optical disc, a magneto-optical disc, or a semiconductor memory. The removable recording medium 927 may be, for example, DVD media, HD-DVD media, Blu-ray (registered trademark) media, or the like. Further, the removable recording medium 927 may be Compact Flash (CF) (registered trademark), a flash memory a secure digital (SD) memory card, or the like. In addition, the removable recording medium 927 may be, for example, an integrated circuit (IC) card having a contactless type IC chip mounted thereon, an electronic apparatus, or the like.

The connection port 923 is a port for direction connection to the information processing apparatus 900. As an example of the connection port 923, there are a universal serial bus (USB) port, an IEEE 1394 port, a small computer system interface (SCSI) port, and the like. An another example of the connection port 923, there are an RS-232C port, an optical audio terminal, high-definition multimedia interface (HDMI) (registered trademark) port, and the like. The information processing apparatus 900 directly acquires various types of data from the external connection apparatus 929 or provides various types of data to the external connection apparatus 929 by connecting the external connection apparatus 929 to the connection port 923.

The communication device 925 is a communication interface configured as a communication device or the like for accessing a communication network (network) 931. The communication device 925 may be, for example, a wired or wireless local area network (LAN), Bluetooth (registered trademark), a communication card for wireless USB (WUSB), or the like. In addition, the communication device 925 may be a router for optical communication, a router for asymmetric digital subscriber line (ADSL), a modem for various communications, or the like. This communication device 925 may transmit/receive signals and the like, for example, according to a predetermined protocol such as TCP/IP between, for example, the Internet and other communication apparatuses. In addition, the communication network 931 connected to the communication device 925 is configured as a network and the like connected in a wired or wireless manner and may be, for example, the internet, a home LAN, infrared communication, radio wave communication, satellite communication, or the like.

An example of the hardware configuration capable of realizing the functions of the information processing apparatus 900 constituting the medical observation system according to an embodiment of the present disclosure has been illustrated above. Each of the aforementioned components may be configured using a universal member or configured as hardware specialized for the function of each component. Accordingly, a hardware configuration to be used may be appropriately changed in response to a technical level when the present embodiment is embodied. Meanwhile, although not illustrated in FIG. 16, various configurations corresponding to the information processing apparatus 900 constituting the medical observation system are included.

Meanwhile, a computer program for realizing each function of the above-described information processing apparatus 900 constituting the medical observation system according to the present embodiment may be manufactured and mounted in a personal computer or the like. In addition, a computer-readable recording medium in which the computer program is stored may also be provided. The recording medium may be, for example, a magnetic disk, an optical disk, a magneto-optical disk, a flash memory, or the like. Further, the aforementioned computer program may be distributed, for example, through a network without using a recording medium. Moreover, the number of computers for executing the computer program is not particularly limited. For example, the computer program may be executed by a plurality of computers (e.g., a plurality of servers) in cooperation.

### 5. APPLICATION EXAMPLE

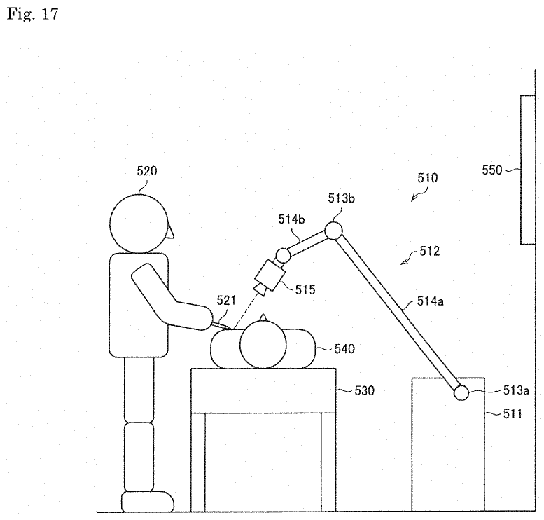

Subsequently, an example of a case in which the medical observation system according to an embodiment of the present disclosure is configured as a microscope imaging system including a microscope unit will be described as an application example of the medical observation system with reference to FIG. 17.

FIG. 17 is an explanatory diagram for describing an application example of the medical observation system according to an embodiment of the present disclosure and represents an example of a schematic configuration of a microscope imaging system. Specifically, FIG. 17 illustrates an example of a case in which a video microscope apparatus for operation including an arm is used as an application example when the microscope imaging system according to an embodiment of the present disclosure is used.

For example, FIG. 17 schematically represents a state of medical treatment using the video microscope apparatus for operation. Specifically, referring to FIG. 17, a state in which a doctor who is an operator (user) 820 performs an operation on an operation target (patient) 840 on an operating table 830 using operating tools 821 such as a scalpel, a tweezer, and forceps, for example, is illustrated. Meanwhile, it is assumed that an operation is a generic term of various medical treatments performed by the doctor who is the user 820 on the patient that is the operation target 840, such as surgeries and examination in the following description. In addition, although a state of a surgery is illustrated as an example of an operation in the example illustrated in FIG. 17, operations using the video microscope apparatus 810 for operation are not limited to surgeries and may be various other operations.

The video microscope apparatus 810 for operation is provided by the operating table 830. The video microscope apparatus 810 for operation includes, a base part 811 that is a base, an arm part 812 extending from the base part 811, and an imaging unit 815 connected to the front end of the arm part 812 as a front-end unit. The arm part 812 includes a plurality of joints 813a, 813b and 813c, a plurality of links 814a and 814b connected by the joints 813a and 813b, and the imaging unit 815 provided at the front end of the arm part 812. In the example illustrated in FIG. 17, although the arm part 812 includes three joints 813a to 813c and two links 814a and 814b for simplification, the number and shape of the joints 813a to 813c and the links 814a and 814b, directions of driving shaft of the joints 813a to 813c, and the like may be appropriately set in consideration of a degree of freedom of positions and postures of the arm part 812 and the imaging unit 815 such that a desired degree of freedom is realized.

The joints 813a to 813c have a function of rotatably connecting the links 814a and 814b, and the operation of the arm part 812 is controlled by driving rotation of the joints 813a to 813c. Here, the position of each component member of the video microscope apparatus 810 for operation means a position (coordinates) in a space designated for operation control and the posture of each component member means a direction (angle) with respect to an arbitrary axis in the space designated for operation control in the following description. In addition, operation (or operation control) of the arm part 812 means operation (or operation control) of the joints 813a to 813c and changing (control of changing) of the position and posture of each component member of the arm part 812 by performing the operation (or operation control) of the joints 813a to 813c.

The imaging unit 815 is connected to the front end of the arm part 812 as a front-end unit. The imaging unit 815 is a unit that acquires an image of an imaging target and may be, for example, a camera and the like capable of capturing moving images and still images. As illustrated in FIG. 17, the postures and positions of the arm part 812 and the imaging unit 815 are controlled by the video microscope apparatus 810 for operation such that the imaging unit 815 provided at the front end of the arm part 812 images the state of an operation site of the operation target 840. Meanwhile, the configuration of the imaging unit 815 connected to the front end of the arm part 812 as a front-end unit is not particularly limited, and the imaging unit 815 may be configured as, for example, a microscope that acquires an enlarged image of an imaging target. Further, the imaging unit 815 may be configured such that it is detachably attached to the arm part 812. According to this configuration, the imaging unit 815 may be appropriately connected to the front end of the arm part 812 as a front-end unit, for example, in accordance with usage application. Meanwhile, an imaging device to which the branching optical system according to the above-described embodiment is applied may be applied, for example, as the imaging unit 815. That is, in the present application example, the imaging unit 815 or the video microscope apparatus 810 for operation including the imaging unit 815 may correspond to an example of a “medical observation apparatus.” Further, although the present description focuses on a case in which the imaging unit 815 is applied as the front-end unit, the front-end unit connected to the front end of the arm part 812 is not necessarily limited to the imaging unit 815.

In addition, a display device 850 such as a monitor or a display is provided at a position facing the user 820. An image of the operation site captured by the imaging unit 815 is displayed as an electronic image on a display screen of the display device 850. The user 820 performs various types of processing while viewing the electronic image of the operation site displayed on the display screen of the display device 850.

According to the above-described configuration, it is possible to perform a surgery while imaging an operation site through the video microscope apparatus 810 for operation.

Meanwhile, the present disclosure is not limited to the above description and the above-described technology according to the present disclosure may be applied without departing from the basic concept of the medical observation system according to an embodiment of the present disclosure. As a specific example, the above-described technology according to the present disclosure may be appropriately applied to systems that enable observation of an affected body part by capturing an image of the affected body part through an imaging device in a desired form in addition to the above-described systems to which an endoscope and a microscope for operation are applied.

An example of a case in which the medical observation system according to an embodiment of the present disclosure is configured as a microscope imaging system including a microscope unit has been described above as an application example of the medical observation system with reference to FIG. 17.

### 6. CONCLUSION

As described above, the medical observation system according to an embodiment of the present disclosure includes a light source that illuminates an affected body part, a branching optical system, a detection unit, an arithmetic operation unit, and a processing unit. The branching optical system separates light from the affected body part into a plurality of polarized lights having different polarization directions. The detection unit individually detects the plurality of polarized lights. The arithmetic operation unit individually calculates speckle contrasts on the basis of detection results of the plurality of polarized lights. The processing unit executes processing with respect to observation of the affected body part on the basis of at least any of calculation results of the speckle contrasts corresponding to the plurality of polarized lights. As a specific example, the processing unit may calculate an average of the calculation results of the speckle contrasts corresponding to the plurality of polarized lights and execute the processing with respect to observation of the affected body part on the basis of the average calculation result.

A speckle contrast individually calculated for each polarized light as described above largely changes a velocity of a motion of the affected body part (i.e., a dynamic range becomes wide) as compared to a speckle contrast calculated without separating the light from the affected body part into polarized lights. According to this characteristic, the medical observation system according to an embodiment of the present disclosure may catch insignificant velocity change in the motion of the affected body part with high sensitivity as compared to a case in which the light from the affected body part is observed without being separated into polarized lights. In addition, according to the medical observation system according to the present disclosure, it is possible to use the light from the affected body part with high efficiency because all speckle contrasts calculated with respect to the plurality of polarized lights separated from the light from the affected body part can be used.

Although suitable embodiments of the present disclosure have been described above in detail with reference to the attached drawings, the technical scope of the present disclosure is not limited to such examples. It will be apparent to those skilled in the art that various modification examples and amendment examples are possible without departing from the scope of the technical spirit described in claims, and it will be understood that these examples also belong to the technical scope of the present disclosure.

Furthermore, the effects described in this specification are explanatory or illustrative and are not limitative. That is, the technology according to the present disclosure may obtain other effects apparent to those skilled in the art from this specification in addition to or instead of the aforementioned effects.

Meanwhile, the following configurations also belong to the technical scope of the present disclosure.

(1) A medical observation system including: a light source configured to illuminate an affected body part; a branching optical system configured to separate light from the affected body part into a plurality of polarized lights having different polarization directions; a detection unit configured to individually detect the plurality of polarized lights; an arithmetic operation unit configured to individually calculate speckle contrasts on the basis of detection results of the plurality of polarized lights; and a processing unit configured to execute processing with respect to observation of the affected body part on the basis of at least any of calculation results of the speckle contrasts corresponding to the plurality of polarized lights.

(2) The medical observation system according to (1), including an endoscope unit including a barrel inserted into a body cavity of a patient, wherein the branching optical system separates the light from the affected body part, acquired by the endoscope unit, into the plurality of polarized lights.

(3) The medical observation system according to (1), including a microscope unit configured to acquire an enlarged image of the affected body part, wherein the branching optical system separates the enlarged image based on light from the affected body part, acquired by the microscope unit, into the plurality of polarized lights.

(4) A medical observation apparatus including: a branching optical system configured to separate light from an affected body part into a plurality of polarized lights having different polarization directions; a detection unit configured to individually detect the plurality of polarized lights; an arithmetic operation unit configured to individually calculate speckle contrasts on the basis of detection results of the plurality of polarized lights; and a processing unit configured to execute processing with respect to observation of the affected body part on the basis of at least any of calculation results of the speckle contrasts corresponding to the plurality of polarized lights.

(5) The medical observation apparatus according to (4), wherein the processing unit combines the calculation results of the speckle contrasts corresponding to the plurality of polarized lights and executes the processing with respect to observation of the affected body part on the basis of a result of the combination.

(6) The medical observation apparatus according to (5), wherein the processing unit calculates an average of the calculation results of the speckle contrasts corresponding to the plurality of polarized lights and executes processing with respect to observation of the affected body part on the basis of a calculation result of the average.

(7) The medical observation apparatus according to (5), wherein the processing unit combines the calculation results of the speckle contrasts corresponding to the plurality of polarized lights on the basis of weights in response to light intensities of the plurality of polarized lights and executes processing with respect to observation of the affected body part on the basis of the result of the combination.

(8) The medical observation apparatus according to (4), wherein, when saturation of signals with respect to detection results of some of the plurality of polarized lights is detected, the processing unit executes processing with respect to observation of the affected body part on the basis of calculation results of the speckle contrasts corresponding to polarized lights from which signal saturation is not detected.

(9) The medical observation apparatus according to any one of (4) to (8), wherein the detection unit includes a plurality of imaging elements, and the plurality of polarized lights separated by the branching optical system from the light from the affected body part are imaged on different imaging elements among the plurality of imaging elements.

(10) The medical observation apparatus according to any one of (4) to (8), wherein the detection unit includes an imaging element, and the plurality of polarized lights separated by the branching optical system from the light from the affected body part are imaged on different areas of a light-receiving surface of the imaging element.

(11) The medical observation apparatus according to (10), wherein the branching optical system includes a plurality of polarization separation elements, the plurality of polarization separation elements separate the light from the affected body part into plurality of polarized lights, and the plurality of polarized lights separated by the plurality of polarization separation elements from the light from the affected body part are imaged on different areas of the light-receiving surface of the imaging element.

(12) The medical observation apparatus according to any one of (4) to (1), wherein the affected body part is blood vessels, and the processing unit executes processing with respect to observation of a blood flow on the basis of at least any of the calculation results of the speckle contrasts corresponding to the plurality of polarized lights.

(13) The medical observation apparatus according to (12), wherein the processing unit generates an image in which the blood flow is presented on the basis of at least any of the calculation results of the speckle contrasts corresponding to the plurality of polarized lights.

(14) A medical observation apparatus including: an arithmetic operation unit configured to individually calculate speckle contrasts on the basis of detection results of a plurality of polarized lights having different polarization directions, separated from light from an affected body part; and a processing unit configured to execute processing with respect to observation of the affected body part on the basis of at least any of calculation results of the speckle contrasts corresponding to the plurality of polarized lights.

(15) A method for driving a medical observation apparatus, using a computer, including: individually calculating speckle contrasts on the basis of detection results of a plurality of polarized lights having different polarization directions, separated from light from an affected body part; and executing processing with respect to observation of the affected body part on the basis of at least any of calculation results of the speckle contrasts corresponding to the plurality of polarized lights.

(16) A program causing a computer: to individually calculate speckle contrasts on the basis of detection results of a plurality of polarized lights having different polarization directions, separated from light from an affected body part; and to execute processing with respect to observation of the affected body part on the basis of at least any of calculation results of the speckle contrasts corresponding to the plurality of polarized lights.

### REFERENCE SIGNS LIST

2, 3 Medical observation system
201 Control unit
203 Imaging unit
207 Input unit
209 Output unit
211 Imaging optical system
213 Branching optical system
215 Imaging element
217 Imaging element
223 Light source
225 Transmission cable
231 Branching optical system
233 Imaging element
301 Control unit
303 Processing unit
305 Arithmetic operation unit
305a First arithmetic operation unit
305b Second arithmetic operation unit
307 Analysis unit
309 Image processing unit
311 Output control unit
313 Detection unit
313a First imaging unit
313b Second imaging unit
317 Output unit

1. A medical observation system comprising:
a light source configured to illuminate an affected body part;
a branching optical system configured to separate light from the affected body part into a plurality of polarized lights having different polarization directions;
a detection unit configured to individually detect the plurality of polarized lights;
an arithmetic operation unit configured to individually calculate speckle contrasts on the basis of detection results of the plurality of polarized lights; and
a processing unit configured to execute processing with respect to observation of the affected body part on the basis of at least any of calculation results of the speckle contrasts corresponding to the plurality of polarized lights.

2. The medical observation system according to claim 1, comprising an endoscope unit including a barrel inserted into a body cavity of a patient, wherein the branching optical system separates light from the affected body part, acquired by the endoscope unit, into the plurality of polarized lights.

3. The medical observation system according to claim 1, comprising a microscope unit configured to acquire an enlarged image of the affected body part, wherein the branching optical system separates the enlarged image based on light from the affected body part, acquired by the microscope unit, into the plurality of polarized lights.

4. A medical observation apparatus comprising:
a branching optical system configured to separate light from an affected body part into a plurality of polarized lights having different polarization directions;
a detection unit configured to individually detect the plurality of polarized lights;
an arithmetic operation unit configured to individually calculate speckle contrasts on the basis of detection results of the plurality of polarized lights; and
a processing unit configured to execute processing with respect to observation of the affected body part on the basis of at least any of calculation results of the speckle contrasts corresponding to the plurality of polarized lights.

5. The medical observation apparatus according to claim 4, wherein the processing unit combines the calculation results of the speckle contrasts corresponding to the plurality of polarized lights and executes processing with respect to observation of the affected body part on the basis of a result of the combination.

6. The medical observation apparatus according to claim 5, wherein the processing unit calculates an average of the calculation results of the speckle contrasts corresponding to the plurality of polarized lights and executes processing with respect to observation of the affected body part on the basis of a calculation result of the average.

7. The medical observation apparatus according to claim 5, wherein the processing unit combines the calculation results of the speckle contrasts corresponding to the plurality of polarized lights on the basis of weights in response to light intensities of the plurality of polarized lights and executes processing with respect to observation of the affected body part on the basis of the result of the combination.

8. The medical observation apparatus according to claim 4, wherein, when saturation of signals with respect to detection results of some of the plurality of polarized lights is detected, the processing unit executes processing with respect to observation of the affected body part on the basis of calculation results of the speckle contrasts corresponding to polarized lights from which signal saturation is not detected.

9. The medical observation apparatus according to claim 4, wherein the detection unit includes a plurality of imaging elements, and
the plurality of polarized lights separated by the branching optical system from light from the affected body part are imaged on different imaging elements among the plurality of imaging elements.

10. The medical observation apparatus according to claim 4, wherein the detection unit includes an imaging element, and
the plurality of polarized lights separated by the branching optical system from light from the affected body part are imaged on different areas of a light-receiving surface of the imaging element.

11. The medical observation apparatus according to claim 10, wherein the branching optical system includes a plurality of polarization separation elements,
the plurality of polarization separation elements separate light from the affected body part into plurality of polarized lights, and
the plurality of polarized lights separated by the plurality of polarization separation elements from light from the affected body part are imaged on different areas of the light-receiving surface of the imaging element.

12. The medical observation apparatus according to claim 4, wherein the affected body part is blood vessels, and
the processing unit executes processing with respect to observation of a blood flow on the basis of at least any of the calculation results of the speckle contrasts corresponding to the plurality of polarized lights.

13. The medical observation apparatus according to claim 12, wherein the processing unit generates an image in which the blood flow is presented on the basis of at least any of the calculation results of the speckle contrasts corresponding to the plurality of polarized lights.

14. A medical observation apparatus comprising: an arithmetic operation unit configured to individually calculate speckle contrasts on the basis of detection results of a plurality of polarized lights having different polarization directions, separated from light from an affected body part; and
a processing unit configured to execute processing with respect to observation of the affected body part on the basis of at least any of calculation results of the speckle contrasts corresponding to the plurality of polarized lights.

15. A method for driving a medical observation apparatus, using a computer, comprising:
individually calculating speckle contrasts on the basis of detection results of a plurality of polarized lights having different polarization directions, separated from light from an affected body part; and
executing processing with respect to observation of the affected body part on the basis of at least any of calculation results of the speckle contrasts corresponding to the plurality of polarized lights.
